(19) United States
(12) Patent Application Publication
(10) Pub. No.: US 2015/0247112 A1
(43) Pub. Date: Sep. 3, 2015

(54) 3D Tissue Culture Devices and Systems

(71) Applicant: KIYATEC INC., Greenville, SC (US)

(72) Inventors: David E. Orr, Piedmont, SC (US); Matthew R. Gevaert, Greenville, SC (US); Chaitra Cheluvaraju, Central, SC (US); Howland E. Crosswell, Greenville, SC (US); Teresa M. DesRochers, Greenville, SC (US)

(21) Appl. No.: 14/637,383

(22) Filed: Mar. 3, 2015

Related U.S. Application Data
(60) Provisional application No. 61/947,359, filed on Mar. 3, 2014.

(51) Int. Cl. C12M 1/32; C12Q 1/68; C12N 5/09; C12M 1/00; G01N 33/50

(52) U.S. Cl. CPC C12M 23/12; C12M 23/22; C12M 23/38; G01N 33/5026; G01N 33/5023; C12N 5/0693; C12Q 1/6886; C12Q 2600/158; C12Q 2600/136; C12Q 2600/16

(57) ABSTRACT

Embodiments disclosed herein are directed to bioreactor devices and systems that allow cultured cells to grow and develop with a three-dimensional aspect. In addition, the bioreactor systems described herein can be used for variation and independent control of environmental factors within the individual sub-wells which can be advantageously co-located in a common chamber. For example, the chemical make-up of a nutrient medium that can flow through a chamber as well as the mechanical force environment within the chamber, including the perfusion flow, shear stress, hydrostatic pressure, and the like, can be independently controlled and maintained for each separate culture chamber of the disclosed systems. Further, the bioreactor systems are designed for easy incorporation into automated systems and minimize or eliminate tubing.

### CROSS-REFERENCE TO RELATED APPLICATIONS

This patent application claims priority to U.S. Provisional Patent Application No. 61/947,359 filed Mar. 3, 2014 and entitled “3D Tissue Culture Devices and Systems.” The entire disclosure of the above-identified priority application is hereby fully incorporated herein by reference

### TECHNICAL FIELD

The subject matter disclosed herein is directed to three-dimensional cell culture devices and uses thereof.

### BACKGROUND

The ability to culture in vitro three-dimensional cellular constructs that mimic natural tissue has proven challenging. There are multiple dynamic biochemical and mechanical interactions that take place between and among cells in vivo, many of which have yet to be fully understood, and yet the complicated in vivo system must be accurately modeled if successful development of engineered tissues in vitro is to be accomplished. Growth of mammalian cells in vitro using traditional culture methods where cells are grown on a flat substrate, such as in a conventional cell culture plate or flask, fails to replicate the complexities cells encounter in vivo. One of the major physical differences relates to the shape and geometry cells acquire when grown on a flat substrate. Growth on two-dimensional surfaces (2D) results in cell flattening and remodeling of the cell and its internal cytoskeleton. Such changes have been shown to alter gene expression. Vergani et al. INT J BIOCHEM CELL BIOL 2004, 36, 1447-1461. Cell flattening also affects nuclear shape, which can lead to differences in gene expression and protein synthesis. Thomas et al. PNAS 2002 99, 1972-1977. This has a significant impact on cell performance and can influence the usefulness of biological assays conducted on 2D cultures. For example, monolayers of cultured cells are though to be more susceptible to therapeutic agents. Bhadriraju & Chen DRUG DISCOV TODAY 2002 7, 612-620; Sun et al. J BIOTECHNOL 2006, 122, 372-381. Furthermore, cell culture on rigid surfaces can enhance cell proliferation but inhibit cell differentiation due to limited cell interactions. Cukierman et al. CURR OPIN CELL BIOL. 2002, 14, 633-639. A more appropriately engineered cell culture environment could improve the predictive accuracy of the drug discovery process and aid in the understanding of cell differentiation and morphogenesis.

### SUMMARY

In one aspect, the embodiments described herein are directed to a bioreactor device comprising one or more chambers and a lid, the lid comprising one or more integrated fluid circuits defined therein. The fluid circuits define inlet and outlet channels and allow perfusion of fluids in and out of the one or more chambers. The chambers may be individual and connected only by placement of the lid, or the chambers may be directly connected to one another. The lid may be made of a single layer or multiple layers. In one example embodiment the lid is made of a bottom layer comprising a soft elastomeric material and the top layer comprises a hard or rigid material. In certain example embodiments, the fluid circuit is defined between the bottom and top layers. The bottom layer may further comprise a molded extension that seals the chamber to the lid. The top or bottom layer may comprise one or more pump interfaces. The top or bottom layer may comprise one or more reservoir interfaces. In certain example embodiments, the chambers are the wells of a commercially available microplate, such as those used for cell culture. The chambers may further comprise an insert, the insert defining one or more sub-wells within the chamber. The sub-wells are used for three-dimensional culturing of cells and may further comprise a cell scaffold for cells to grow on, into, or within. In certain example embodiments, the bioreactor device has the footprint of a standard microplate for use with existing devices, such as spectrometry devices.

In another aspect, the embodiments described herein are directed to adapters that can convert individual microplate wells into perfused three-dimensional cell culture sub-wells. The adapter comprises a lid defining an inlet and outlet channel that can be connected to a media reservoir and/or pump to allow perfusion through the adapter. The adapter further comprises an insert that defines one or more sub-wells within the microplate well for cell culture. The sub-wells may further comprise a cell scaffold.

In another aspect, the embodiments described herein are directed to bioreactor devices comprising a soft elastomeric layer defining one or more inlet and outlet fluid channels and one or more holes that traverse the soft layer completely, and a rigid top layer comprising one or more access ports to align with the one or more cell culture chambers. The one or more cell access ports may function as a cell culture chamber. The cell access ports further comprise one or more channels within the walls of the access port to align with the one or more inlet and outlet fluid channels of the soft layer. In certain example embodiments, the one or more channels in the walls of the one or more access ports may enter the chamber at different heights.

In another aspect, the embodiments described herein are directed to bioreactor devices comprising a hard bottom layer defining one or more cell culture chambers, the one or more cell culture chambers comprising an inlet opening and outlet opening at a base of the cell culture channel, and a soft elastomeric top layer for mounting to the hard layer, the soft elastomeric layer defining one or more openings to receive the one or more cell culture chambers, the soft elastomeric layer further defining inlet and outlet fluid channels that align with the inlet and outlet opening in the base of the cell culture chamber.

In another aspect, the embodiments disclosed herein are directed to methods of culturing one or more cell types in a perfused three-dimensional environment comprising seeding the one or more cells in a bioreactor device disclosed herein and perfusing the appropriate cell culture media through the chamber and/or sub-wells of the device.

In yet another aspect, the embodiments disclosed herein are directed to method of assessing responsiveness to therapeutic agents comprising culturing in a perfused three-dimensional environment a plurality of cell samples in a plurality of sub-wells of the bioreactor devices disclosed herein, perfusing to each chamber a culture media comprising the same or a different therapeutic agent, or concentration of therapeutic agent, and measuring the responsiveness of each cell sample to the corresponding therapeutic agent or concentration of therapeutic agent. In certain example embodiments, the cell samples can be derived from subject biopsy samples and used to select an appropriate therapeutic for the subject based on the measured responsiveness. In certain example embodiments, the biopsy sample is a cancer biopsy sample. In certain other embodiments, cells derived from the biopsy sample are cultured in a three-dimensional environment containing one or more non-cancer cell types such epithelial cells, fibroblast, adipocytes, endothelial cells, macrophages, T-cells, B-cells or a combination thereof.

### BRIEF DESCRIPTION OF DRAWINGS

FIG. 1 is a diagram depicting a multi-chamber bioreactor device with integrated fluid circuits, in accordance with certain example embodiments.

FIG. 2 is a diagram depicting an alternative view of a multi-chamber bioreactor device with integrated fluid circuits, in accordance with example embodiments.

FIG. 3 is a photograph of a multi-chamber bioreactor device with integrated fluid circuits, in accordance with certain example embodiments.

FIG. 4 is a photograph providing a focused view on a multi-well chamber of a bioreactor device with integrated fluid circuits, in accordance with certain example embodiments.

FIG. 5 is a photograph of a bottom view of a multi-chamber bioreactor device with integrated fluid circuits, in accordance with certain example embodiments.

FIG. 6 is a photograph of a top view of a multi-chamber bioreactor device with integrated fluid circuits, in accordance with certain example embodiments.

FIG. 7 is a photograph of a frontal angle view of a multi-chamber bioreactor device with integrated fluid circuits, in accordance with certain example embodiments.

FIG. 8 is a diagram of a portion of a lid inserted into a chamber of a bioreactor device, in accordance with certain example embodiments.

FIG. 9 is a diagram of a portion of a lid inserted into a chamber of a bioreactor device, the lid comprising a domed bubble trap, in accordance with certain example embodiments.

FIG. 10 is a diagram of a portion of a lid inserted into a chamber of a bioreactor device, the lid comprising a pyramidal bubble trap, in accordance with certain example embodiments.

FIG. 11 is a diagram of a portion of lid inserted into a chamber of a chamber of a bioreactor device, the lid further comprising tube extensions that extend from the lid into an insert in the bottom of the chamber, in accordance with certain example embodiments.

FIG. 12 is a diagram of a portion of a lid inserted into a chamber of a bioreactor device, with an insert retained within the bottom portion of the lid, in accordance with certain example embodiments.

FIG. 13 is a diagram of a portion of a lid inserted into a chamber of a bioreactor device, with an insert retained across a bottom portion of the lid, in accordance with certain example embodiments.

FIG. 15 is a diagram depicting a chamber with a single inlet and single outlet (A), a chamber with a single inlet and single outlet (B), wherein the single inlet has a larger diameter/width than the outlet, and a chamber with multiple inlets and a single outlet (C), in accordance with certain example embodiments.

FIG. 16 is a diagram depicting a fluid circuit with an analytical window defined within the fluid circuit, in accordance with certain example embodiments.

FIG. 17 is a diagram depicting two individual rounded chambers annealed together, in accordance with certain example embodiments.

FIG. 18 is a diagram depicting a single oval-shaped chamber, in accordance with certain example embodiments.

FIG. 19 is a diagram depicting a top view of a rounded chamber with a flat wall surface (A), and a side view (B) of the rounded chamber with a flat wall surface, the flat wall surface comprising an opening and a tab for assisting in locking two chambers together, in accordance with certain example embodiments.

FIG. 20 is a diagram depicting a top view of a double-walled rounded chamber defining a well within a well arrangement, in accordance with certain example embodiments.

FIG. 21 is a diagram depicting a side view (A), a top view (B), and a cross-sectional view (C) of a series of chambers connected by a bridge with a rounded edge, in accordance with certain example embodiments.

FIG. 22 is a diagram depicting a top view (A) and side view (B) of a chamber with an integrated imaging window, in accordance with certain example embodiments.

FIG. 23 is a diagram depicting a cross sectional view of a lid, chamber, and insert of a bioreactor device, in accordance with certain example embodiments.

FIG. 24 is a diagram depicting a series (A-F) of alternative sub-well arrangements within a chamber formed using an insert, in accordance with certain example embodiments.

FIG. 25 is a diagram depicting a series (A-C) of alternative sub-well arrangements within a chamber formed using an insert, in accordance with certain example embodiments.

FIG. 26 is a diagram depicting a chamber with void space around formed sub-wells filled with a hydrogel or porous substrate (A), and an insert cut from a porous filter to define two sub-wells within a chamber (B), in accordance with certain example embodiments.

FIG. 27 a diagram depicting a chamber with retaining blocks for holding inserts, in accordance with certain example embodiments.

FIG. 28 is a diagram depicting a chamber with an insert defining two sub-wells with the remaining void space filled with hydrogel or porous substrate, in accordance with certain example embodiments.

FIG. 29 is a diagram depicting an insert defining a well within a well arrangement, in accordance with certain example embodiments.

FIG. 30 is a set of photographs (A-B) of adapters for converting a single well of a multi-well culture plate into a 3D culture chamber, in accordance with certain example embodiments.

FIG. 31 is a set of photographs of an adapter with an insert defining a single well (A) and an insert defining two sub-wells (B), in accordance with certain example embodiments.

FIG. 32 is a set of photographs providing a side view (A), a first top view (B), a bottom view (C), and second top view (D) of an adapter inserted into a well of a micro-well culture plate, in accordance with certain example embodiments.

FIG. 33 is a diagram depicting a bioreactor device with a fluid channel and chamber defined in a soft elastomeric bottom layer with a hard top layer defining an inlet or access window, in accordance with certain example embodiments.

FIG. 34 is a diagram depicting a top view (A) and bottom view (B) of a bioreactor device with a hard bottom layer defining a plurality of culture chambers and a soft elastomeric top layer with a series of openings for fitting over the culture chambers, in accordance with certain example embodiments.

FIG. 35 is a table showing example cell line co-cultures used to generate microtumors, in accordance with certain example embodiments.

### DETAILED DESCRIPTION OF THE EXAMPLE EMBODIMENTS

### Overview

Embodiments disclosed herein are directed to bioreactor devices and systems that allow cultured cells to grow and develop with a three-dimensional aspect. In addition, the bioreactor systems described herein can be used for variation and independent control of environmental factors within the individual sub-wells which can be advantageously co-located in a common chamber. For example, the chemical make-up of a nutrient medium that can flow through a chamber as well as the mechanical force environment within the chamber, including the perfusion flow, shear stress, hydrostatic pressure, and the like, can be independently controlled and maintained for each separate culture chamber of the disclosed systems. Further, the bioreactor systems are designed for easy incorporation into automated systems and minimize or eliminate tubing. In certain example embodiments, the bioreactor devices provide small culture volumes within chambers that allow for direct analytics to be done on or by the device, allow full thickness imaging, and permit continuous monitoring and measurement of the growth of a cell culture.

In one aspect, the systems disclosed herein can be utilized for culturing product cells for medical use, for example, for transplant to a patient or for manufacture of a protein product, such as a biopharmaceutical. According to this embodiment, cells can be grown in an environment that includes the biochemical products of different cell types, at least some of which may be necessary for the growth and development of the desired cells. However, cell types can be maintained in a physically isolated state during their growth and development. As such, possible negative consequences due to the presence of aberrant or undesired cell types in the desired product cells can be avoided.

In another aspect, the systems disclosed herein can be used to more closely study the biochemical communication between different cell types and the influence of this biochemical communication on the growth and development of cells. As the local environment within each culture chamber of the system can be independently controlled while biochemical communication between chambers, or sub-wells within chambers, can be maintained, information regarding the growth and development of cells and the influence of the local environment on that growth and development can be examined through the use of the devices and systems disclosed herein.

In another aspect, the devices disclosed herein can be used to study the triggering mechanisms involved in stem cell differentiation or to provide isolated, differentiated cells for implantation. For example, undifferentiated stem cells can be located in a first chamber or well, and one or more types of feeder cells can be located in an adjacent chamber(s) or sub-wells.

In another aspect, the devices disclosed herein can be used to study the resistant cell populations that are either residual as a result of, or caused by, exposure of cells to a therapeutic agent. For example, primary cells of a single type or of multiple types obtained from a patient's tumor can be located in a well or chamber of the device, exposed to therapeutic agents that affect the cells resulting in a relative change in the ratio of certain residual or emergent cell populations to other cell populations, and maintain the residual or emergent cells for continued study. For example an assessment of the effect of a different therapeutic agent, or the same therapeutic agent over multiple exposures.

In another aspect, the devices disclosed herein can incorporate primary cells obtained from a patient, expose the cells to therapeutic agents, enable measurement of an effect of the therapeutic agent and be a source for information that is used to draw conclusions about the likelihood of an effect of the therapeutic agent that the patient would experience if actually administered the therapeutic agent or similar or related agents. By way of specific example, such conclusions can be drawn about the effect on either the original cell populations obtained from the patient, and/or about the effect on resistant cell populations.

In yet another aspect, the devices disclosed herein can be used to establish microtumor models. Tumors exist in a complex microenvironment composed of many cell types. For example, the microenvironment of the lung consists primarily of epithelial cells, fibroblasts, and endothelial cells in contact with ECM. The bioreactor devices disclosed herein can be used to develop microtumor models that more accurately reflect a cancer's microenvironment. The cancer cells may be obtained from established cancer cell line or obtained directly from patient biopsy samples. The cancer cells are then grown in combination with one or more other cell types to more closely replicate the appropriate microenvironment. Such microtumor models may then be used to assess the efficacy of different therapeutic agents. Where the sample is derived directly from a patient sample, such screening can be used to guide the course of therapeutic treatment by selecting the therapeutic agent or therapeutic agents that has shown the greatest efficacy in the context of the microtumor model.

In another aspect, the devices disclosed herein integrate micropump automation for perfusion flow and provide multiple replicates in a device footprint conducive to interfacing with existing instrumentation. This includes, for example, automated pipetting stations as well as spectrometer plate readers and microscopes for in situ monitoring and quantitative evaluation of culture conditions and outcomes. Each individual replicate on the device may represent an independent, closed system, with an integrated fluidic circuit that seamlessly transitions into the inlet and outlet ports contained on the device. Each replicate may be positioned in parallel fashion and each driven by its own micropump head. The devices disclosed herein may be utilized in a rack system that can accommodate multiple devices in a space-saving stacked configuration.

Turning now to the drawings, in which like numerals represent like (but not necessarily identical) elements throughout the figures, example embodiments are described in detail.

### Description of Example Embodiments

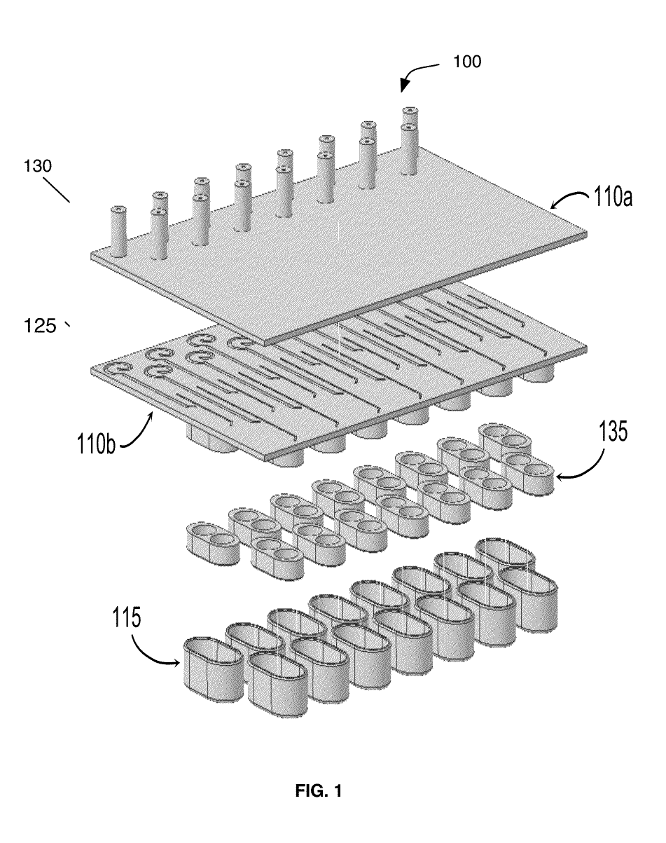

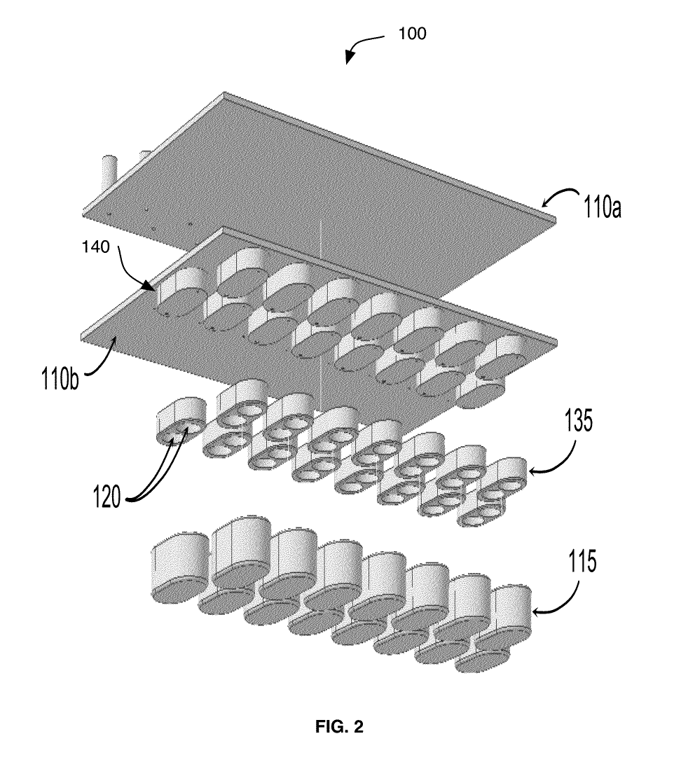

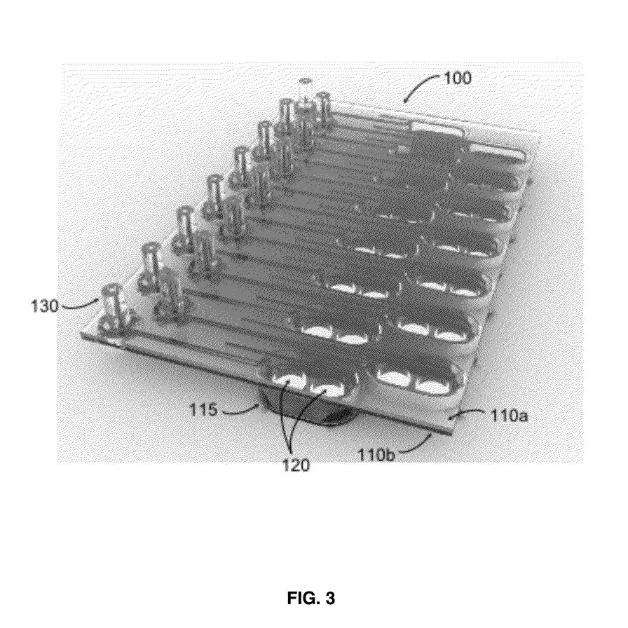

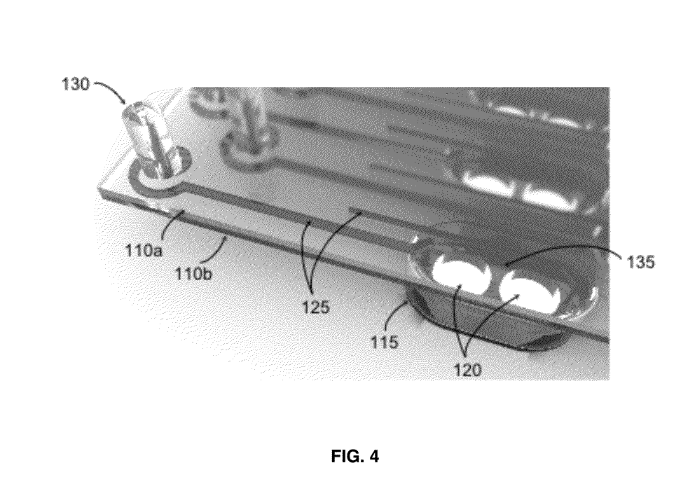

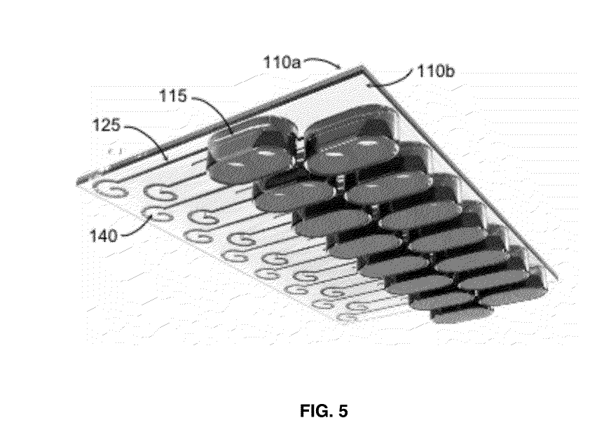

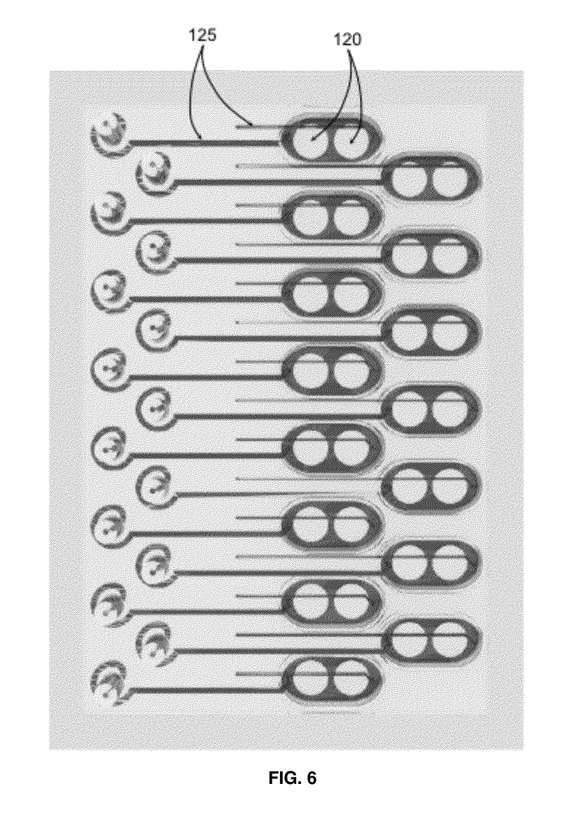

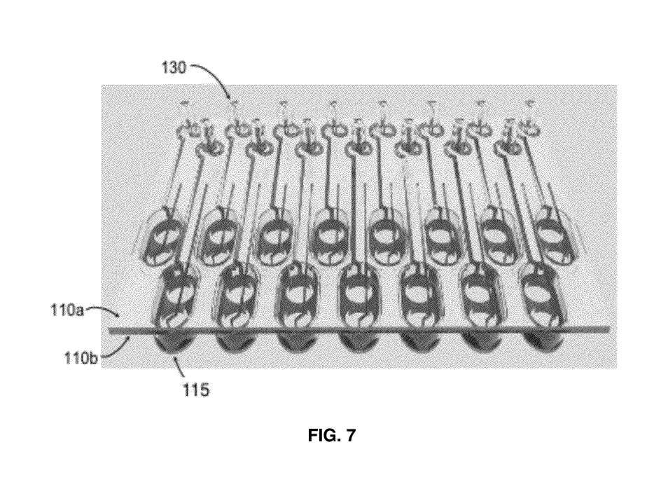

Referring to FIGS. 1-7, multiple views of a bioreactor device 100 in accordance with certain example embodiments are provided. The bioreactor device 100 comprises one or more chambers 115 and a lid 110. The bioreactor device 100 may further comprise one or more inserts 135 defining one or more sub-wells 120 within the one or more chambers 115.

The lid 110 may comprise a single layer, or multiple layers, such as 110a and 110b. As show in the example embodiments of FIGS. 1-7, the lid comprises a top layer 110a and a bottom layer 110b. The top layer 110a may comprise a soft elastomeric material, a hard or rigid material, or a combination thereof. In certain example embodiments, the top layer 110a is constructed of a hard material. The bottom layer may comprises a hard material, a soft elastomeric material or a combination thereof. In certain example embodiments, the lid comprises two soft layers. The two soft layers may be made from the same or different materials. In certain other example embodiments, the lid may comprise two rigid layers. The two rigid layers may be made from the same or different materials. In certain example embodiments, and as show in the example embodiments of FIGS. 1-7, the lid may comprise a soft elastomeric bottom layer 110b and a hard top layer 110a. The soft elastomeric material may have a Young's modulus of approximately 250 KPa to approximately 10 MPa. Example soft materials include PDMS silicone, thermoplastic elastomers, polyurethane, and other soft elastomers. The hard or rigid material may have a Young's modulus of approximately 1 GPa to approximately 4 GPa. Example hard materials include polystyrene, polycarbonate, cyclic olefin copolymer, cyclic olefin polymer, or similar other rigid materials. The various lid layers 110a and 110b may be sealed together using heat seals, adhesive seals, or plasma treatment to allow chemical bond formation between layers.

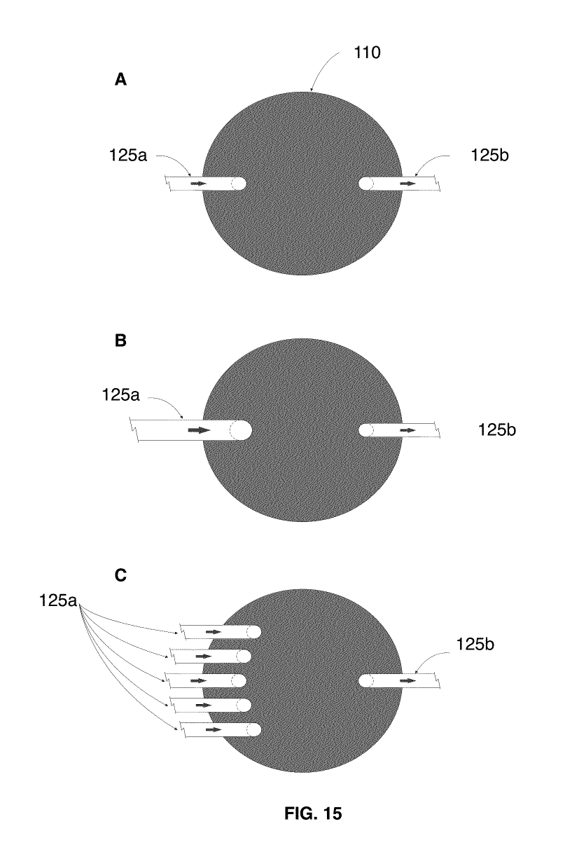

In certain example embodiments, the lid further comprises fluid circuits 125 defined therein. See FIGS. 1-7. The fluid circuit 125 may be defined within a single layer, or between layers of the lid 110a and bottom layer 110b using standard molding techniques to create the desired fluid flow. For example, the fluid circuit 125 may only direct fluid flow to an individual incubation chamber 115, or the fluid circuit may be defined in the lid layers 110a and/or 110b to direct fluid flow to multiple incubation chambers 115. The fluid circuit 125 may further direct media to specified porous regions of inserts within the chambers 115 or to the location of a defined culture construct or scaffold. In certain example embodiments, the bottom soft layer 110b defines an inlet channel 125a and an outlet channel 125b for each chamber 115. In certain example embodiments, the inlet channel 125a and outlet channel 125b may be the same size. See. FIG. 15a. In certain other example embodiments, the inlet channel 125a may be of a greater width or diameter than the outlet channel 125b. See FIG. 15B. Such an embodiment may be used to increase chamber pressure, for example, to mimic tumor physiology. Likewise, the number of inlet and/or outlet ports may be used to increase chamber pressure. In certain example embodiments, there are multiple inlet ports and a smaller number of outlet ports compared to the number of inlet ports. See FIG. 15C. In another example embodiment, there are multiple outlet ports and a smaller number of inlet ports compared to the number of inlet ports.

The fluid circuits 125 may have a width of approximately 1 μm to approximately 10 mm. The fluid circuits may have a height of approximately 1 μm to approximately 10 mm. The fluid circuit may have a height to width ratio of approximately 1 to approximately 10,000.

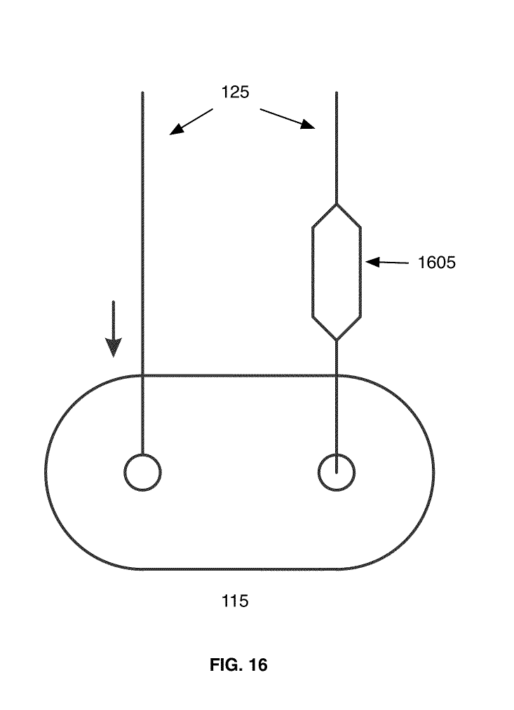

Referring to FIG. 16, in accordance with certain example embodiments, the flow circuit 125 may further comprise an analytical window 1605 in series with the flow coming out of a chamber 115. The analytical window allows for in situ spectrometry, colorimetric, fluorimetric, and bioluminescent readings of the media flow coming out of a chamber 115.

The lid 110 may further comprise one or more external pump interfaces 130. The external pump interface 130 may connect to a pump device (not shown), such as a micropump device, for introducing culture media from an external media reservoir (not shown) into the device 100. The pump head of the pump device may connect above or beneath the device 100. The pump device may be connected to a fluid circuit 125 or individual chamber 115 or lid via tubing into a port located in the fluid circuit 125 or lid 110a. In certain example embodiments the external pump interface 130 is defined on a hard layer 110a.

The lid 110 may further define other ports or openings for accessing the contents of an incubation chamber 115 or well 120. For example, an access port may be defined in the lid 110 to provide access to a chamber 115 or well 120 for sampling, such as a biopsy port to access a 3D cell/tissue construct and take a needle biopsy for DNA/RNA/protein analysis. The lid 110 may define sensor ports for inserting sensors, such as, but not limited to oxygen sensors, temperature sensors, and pressures sensors for monitoring the internal biochemical and biophysical parameters of an incubation chamber 115 or well 120.

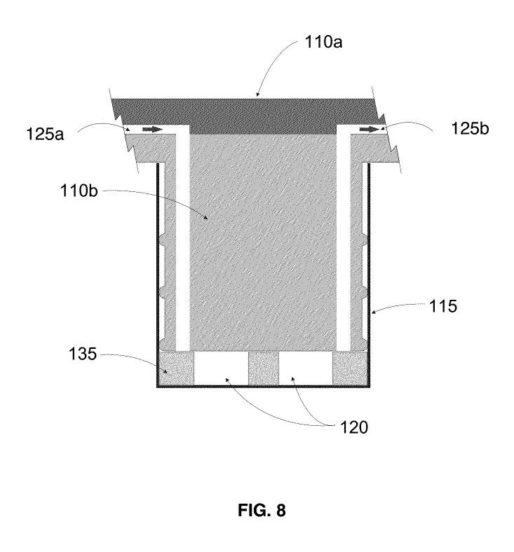

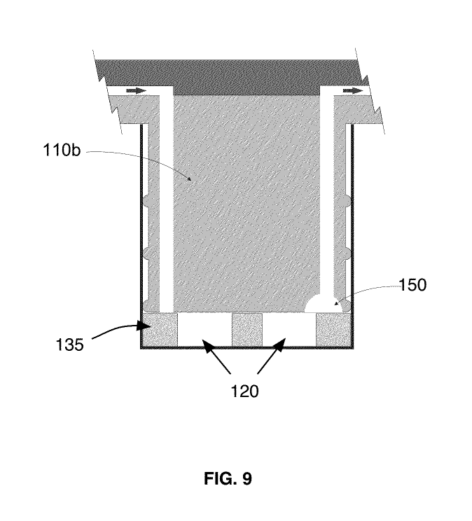

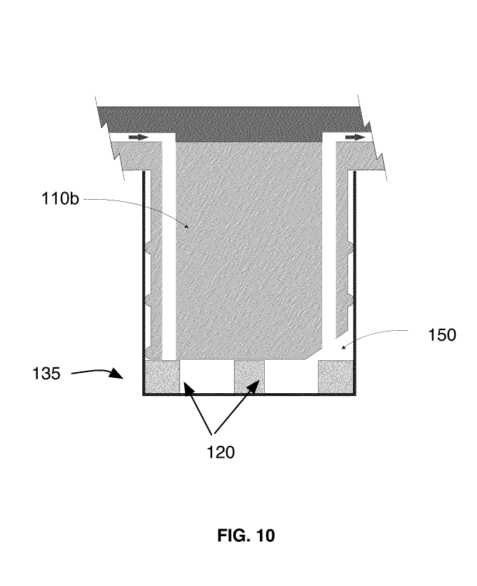

The lid 110a may comprise a plug extension 140 that extends into the incubation chamber 115 to seal the incubation chamber 115. The plug extension 140 may be a continuous or molded part of the bottom layer 110b. The input channel and output channel of the fluid circuit 125 may be further defined in the plug extension 140 to direct media to specified regions of the inserts 135 or sub-wells 120. See FIG. 8. In certain example embodiments, the lid 110b may further include an air bubble trap 150. The lid may be configured so that the bubble trap is next to an outlet channel for evacuation of the trapped air bubbles under normal flow conditions. For example, the air bubble trap may be a domed or pyramidal bubble trap molded into the lid to capture air bubbles and position the air bubbles near an outlet channel for removal. FIG. 9 provides an example domed bubble trap. FIG. 10 provides an example of a pyramidal bubble trap. In certain other example embodiments, the air bubble trap may be an angled lid bubble trap. In certain example embodiments, the lid may further define additional ports to remove air bubbles. For example, the lid may define a tube port for connecting to tubing hooked to a vacuum source. The evacuation port may be positioned next to the air bubble traps discussed above.

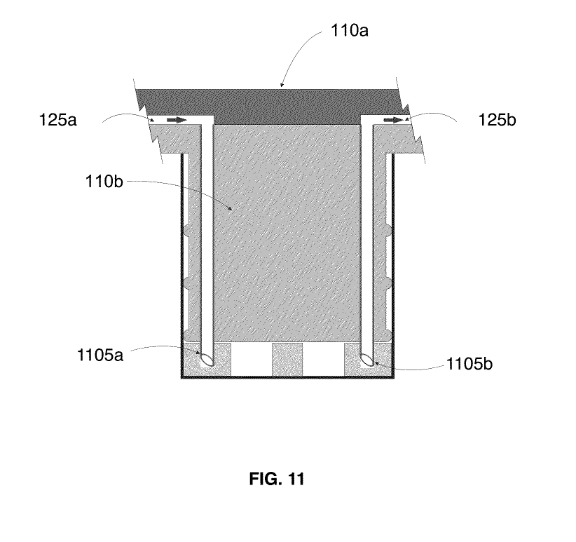

Referring to FIG. 11, in accordance with certain example embodiments, the lid 110b may further comprise tubing extensions 1105a and 1105b that extend tubing further into the chamber 115 or sub-well 120 to more precisely direct fluid flow within the chamber 115, or sub-well 120. The tubing may be square, round, or other suitable shape and may have an end cut that is perpendicular or angled with respect to the direction of flow through the tubing. In certain example embodiments, the tubing may be extended into a sized hole pre-formed in a porous substrate material.

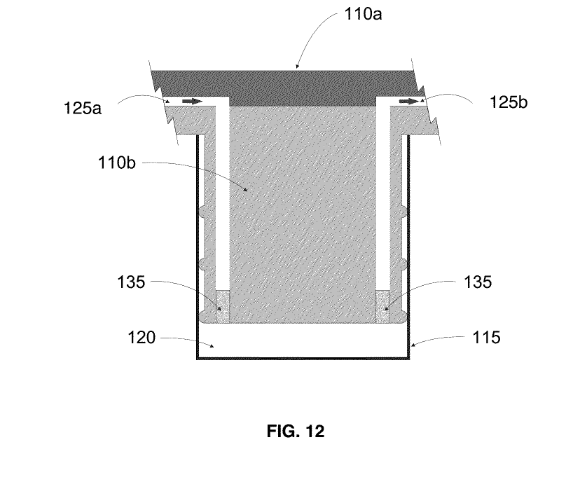

Referring to FIG. 12, in accordance with certain example embodiments, the lid 110b may be designed to accept a portion of the insert 135 within the inlet channel 125a and outlet channel of the fluid circuit 125b. For example, the insert 135 may be located in the bottom portion of the input channel 125a and output channel 125b, rather than in the bottom of the chamber 115. In certain example embodiments, the insert material may further extend to and/or fill the bottom portion of the well.

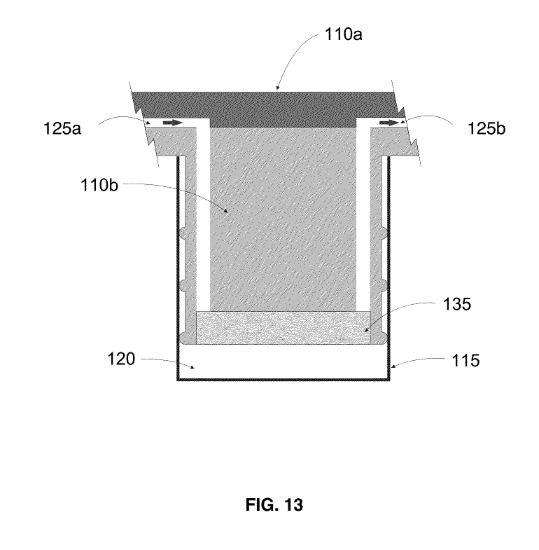

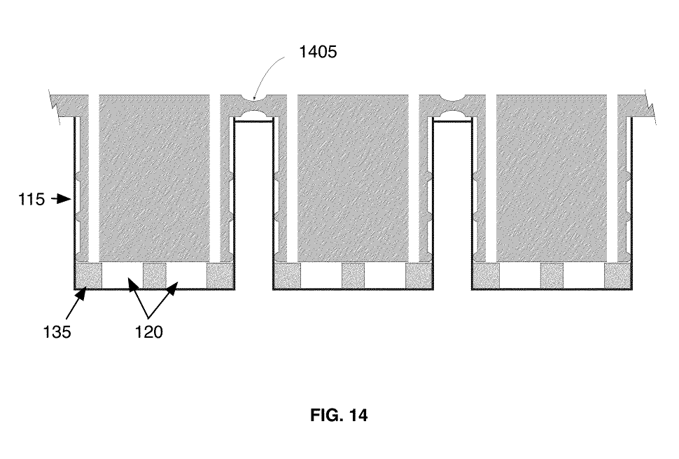

Referring to FIG. 13, in accordance with certain example embodiments, the lid 110b may comprise an extension to hold insert 135, the extension extending beyond the end of an input channel 125a and output channel 125b for a particular chamber 115. For example, the insert may be located inside the extension and covering the bottom surface of the lid 110b. In certain example embodiments, the insert material may further extend to and/or fill the bottom portion of the well.

Referring to FIG. 14, in accordance with certain example embodiments, the lid 110 may be made with one or more partitions 1405. The partitions 1405 may be molded into an elastomeric lid 110 allowing easier application during the sealing process. Partitions may be physically cut to gain access to an individual chamber 115 when necessary, thereby preventing a user from having to open all chambers or sub-wells at the same time. In certain example embodiments, the partitions may also be molded with perforations to allow tearing along the partition by hand.

The bioreactor device 100 comprises one or more chambers 115. The one or more chambers 115 may be molded together to define a base (not shown). As shown in the example embodiments of FIGS. 1-7, the chambers 115 are individual and are not connected together. In certain example embodiments, the one or more chambers 115 may be the wells of a microplate, such as a commercially available microplate. In certain example embodiments, the microwell plate is a 6, 12, 24, 38, 96, or 384 well-sized microplate. Individual chambers 115 may be any shape. For example the chambers 115 may be circular, oval, square, rectangular, or cylindrical in shape. The chambers may be connected to one another via the lid 110 as described in further detail below, or may be connected directly to one another by ports defined directly within the chamber walls. It is to be understood that embodiments that depict chambers with inlet and/or outlet ports extending from the chamber walls, may also be used in embodiments where the inlet/outlet ports are removed and the chambers are connected by a lid 110 with a fluid circuit 125 defined therein. In certain example embodiments, the inlet an outlet ports may comprise barbed tubing connectors or standard luer connectors or other similar connectors. In certain example embodiments the inlet and outlet ports enter the chamber near the top, middle, or bottom of the incubation chamber 115. In certain example embodiments, the inlet and outlet ports enter the incubation chamber near the top to prevent bubbles from being trapped in the chamber 115. In another example embodiment, a first port enters the chamber near the top on the chamber wall, and a second port enters the chamber near the bottom of the chamber wall. In certain example embodiments, the base and chamber 115 is used in a vertical integration. In accordance with such example embodiments, the outlet ports may enter at any point in the chamber wall.

The number of inlet and/or outlet ports may be used to increase chamber pressure, for example, to mimic tumor physiology. In certain example embodiments, there are multiple inlet ports and a smaller number of outlet ports compared to the number of inlet ports. See FIG. 15C. In another example embodiment, there are multiple outlet ports and a smaller number of inlet ports compared to the number of inlet ports. The size of the ports may be used to increase chamber pressure. For example an inlet port greater in size than a corresponding outlet port can result in an increase in chamber pressure. See FIG. 15A. In certain example embodiments, the base, like the lid 110a, may include openings for inlet and outlet ports as well as the ports described above.

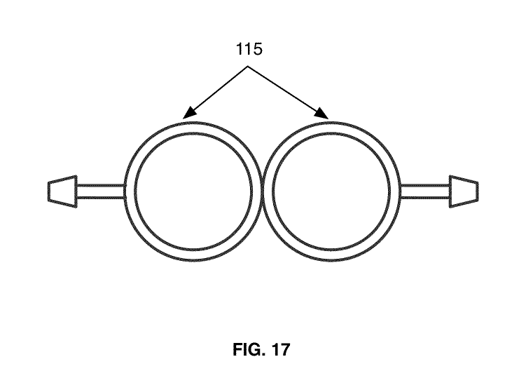

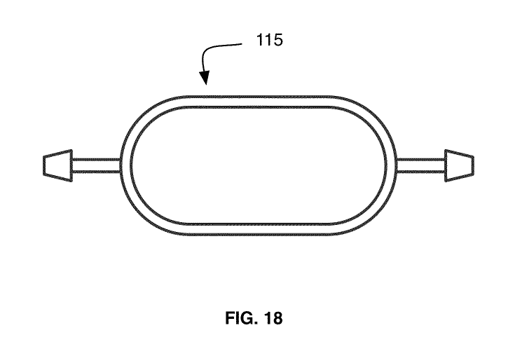

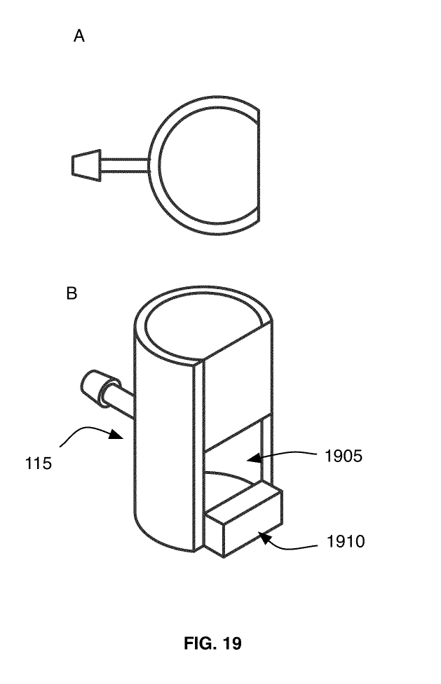

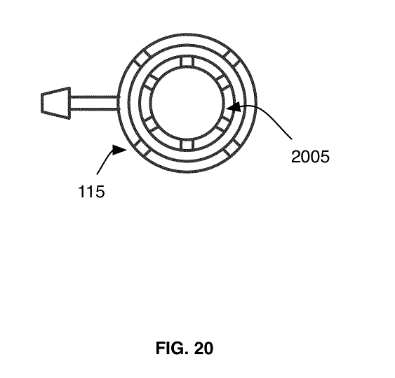

Referring to FIG. 17, in accordance with certain example embodiments, the chamber 115 may be circular in shape. One or more circular chambers may be directly adhered together. Referring to FIG. 18, in accordance with certain example embodiments, the chamber 115 may be oval in shape. Referring to FIG. 19, the chamber 115 may comprise a partially circular wall and a flat wall face. In certain example embodiments, the flat wall face may define an opening 1905 in a portion of the flat wall face to allow for fluid communication between chambers. In certain example embodiments a membrane may be placed in the opening between two chambers. The bottom of the chamber may have an extension 1910 to mate with an opening in base of second chamber to facilitate connecting the two chambers. Referring to FIG. 20, in accordance with certain example embodiments, the chambers 115 may define a comprise an inner wall 2005 defining a well within a well arrangement.

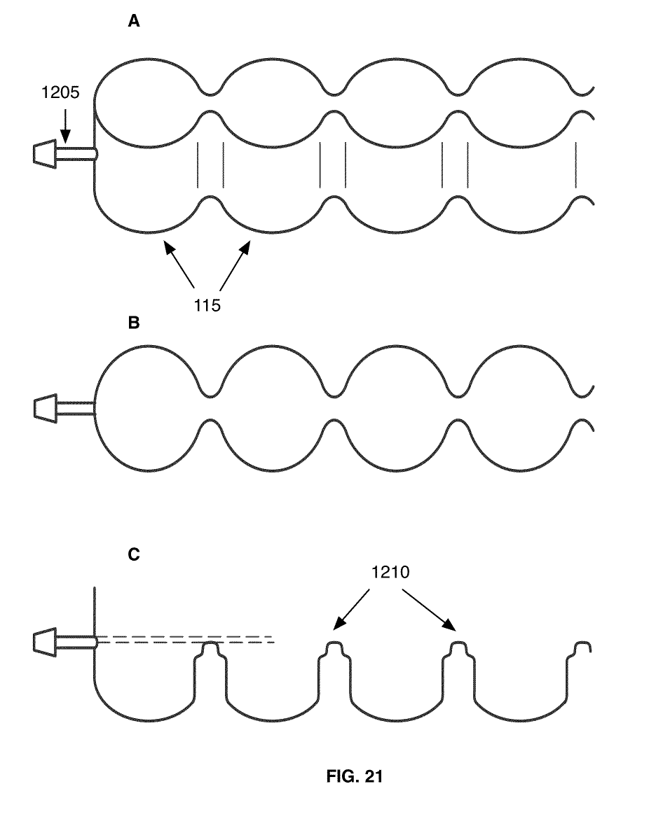

In certain example embodiments, multiple chambers may be formed together as an integrated unit. Referring to FIG. 21, in accordance with certain example embodiments, the one or more chambers 115 may comprise multiple chambers formed as a single unit. Terminal chambers on either end of the row of chamber may be connected to an inlet port/channel 1205 or outlet port/channel respectively, with the intervening chambers connected to once another with a bridge comprising rounded edge connectors 1210 to allow for laminar flow through the chambers. In certain example embodiments, the inlet port/channel is centered at the level of the rounded edges height. See FIG. 21C

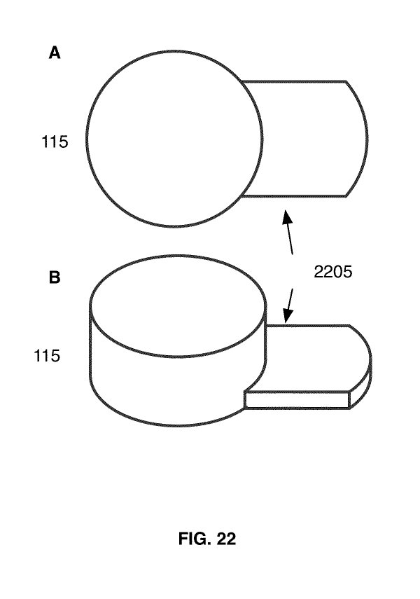

Referring to FIG. 22, in accordance with certain example embodiments, the chamber 115 may further comprise an analytical imaging window 2205 just off of the chamber 115 to accommodate spectrometry reading of biochemical assays. The analytical imaging window is a fraction of the height of the chamber. In certain example embodiments, the analytical imaging window is approximately 50 μm to approximately 1.25 mm in height. The height of the analytical imaging window is based on the specifications and limitations of the analysis or imaging method used which may change over time and to which the dimensions of the analytical imaging window can adapt.

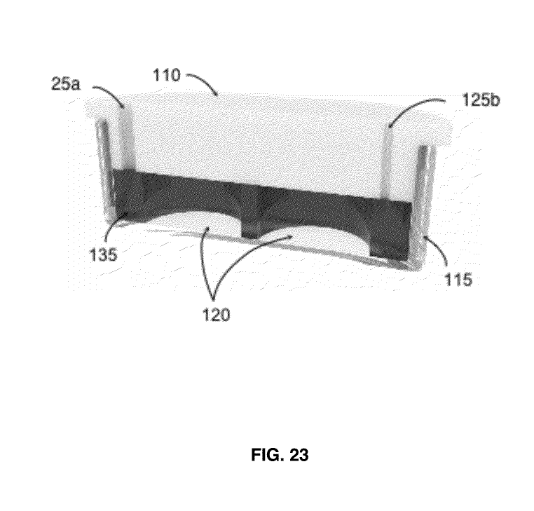

Referring to FIG. 23, in accordance with certain example embodiments, each incubation chamber may be connected to the inlet channel 125a and outlet channel 125b of a flow circuit 125. In certain example embodiments, each incubation chamber 115 may be connected to a separate flow circuit 125. In certain other example embodiments, a flow circuit 125 may be connected to two or more incubation chambers 115 in series. In certain other example embodiments, a flow circuit 125 may be connected to two or more incubation chambers 115 in parallel with flow into the two or more chambers being controlled by a valve device (not shown). The lid 110 covers and seals all incubation chambers 115 to form a closed system. The lid 110 may be made from a transparent material to accommodate microscopy, spectroscopy, and other imaging based analysis. In certain example embodiments, the device 100 is sized to fit within standard multi-well culture plate footprints to accommodate use in standard instrumentation, such as, but not limited to, spectroscopy plate readers. The lid 110 may form the larger physical footprint when the incubation chambers 115 are individual in form and not connected.

The number of chambers 115 may vary. The chambers may be clear and transparent to accommodate microscopy, spectroscopy and other imaging based analysis. Multiple chamber 115 arrangements are possible. In certain example embodiments there is one chamber 115 per flow circuit 125. In another example embodiments, the chambers 115 may be arranged in a staggered or aligned formation. In another example embodiment, one or more chambers 115 may be connected to a common flow circuit 125. The chamber portion of the base may be made out of the same material or a different material than the remainder of the base. The incubation chambers 115 may be made from cyclic olefin copolymer (COC), cyclic olefin polymer (COP), polystyrene, polycarbonate, polypropylene, polyethylene, or other similar materials. The chamber(s) 115 may be formed in various shapes. In certain example embodiments, the chamber 115 is rounded, square, rectangular, or oval in shape.

In certain example embodiments, the bioreactor device comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 individual chambers. In certain example embodiments, each chamber has its own fluid circuit each drive by its own micropump head.

In certain example embodiments the chamber 115 volume may be approximately 150 μL to approximately 250 μL, approximately 150 μL to approximately 225 μL, approximately 150 μL to approximately 200 μL, or approximately 150 μL to approximately 175 μL. In certain example embodiment, the chamber volume may be approximately 50 μL to approximately 400 μL, approximately 50 μL to approximately 375 μL, approximately 50 μL to approximately 350 μL, approximately 50 μL to approximately 300 μL, approximately 50 μL to approximately 275 μL, approximately 50 μL to approximately 250 μL, approximately 50 μL to approximately 225 μL, approximately 50 μL to approximately 200 μL, approximately 50 μL to approximately 175 μL, approximately 50 μL to approximately 150 μL, approximately 50 μL to approximately 125 μL, approximately 50 μL to approximately 100 μL, or approximately 50 μL to approximately 75 μL. In certain other example embodiments, the chamber volume may range from 25 μL to 20 mL, from approximately 25 μL to approximately 10 mL, from approximately 25 μL to approximately 5 mL, from approximately 25 μL to approximately 1 mL, from approximately 25 μL to approximately 750 μL, from approximately 25 μL to approximately 500 μL, from approximately 25 μL to approximately 250 μL.

In certain example embodiments, the chamber has a height of approximately 3.25 mm. In certain other example embodiments, the chamber has a height of approximately 2 mm to approximately 5 mm, approximately 2 mm to approximately 4 mm, or approximately 2 mm to approximately 3 mm. In certain other example embodiments, the chambers have a height of approximately 1 mm to approximately 18 mm, approximately 1 mm to approximately 17 mm, approximately 1 mm to approximately 16 mm, approximately 1 mm to approximately 15 mm, approximately 1 mm to approximately 14 mm, approximately 1 mm to approximately 13 mm, approximately 1 mm to approximately 12 mm, approximately 1 mm to approximately 11 mm, approximately 1 mm to approximately 10 mm, approximately 1 mm to approximately 9 mm, approximately 1 mm to approximately 8 mm, approximately 1 mm to approximately 7 mm, approximately 1 mm to approximately 6 mm, approximately 1 mm to approximately 5 mm, approximately 1 mm to approximately 4 mm, approximately 1 mm to approximately 3 mm, or approximately 1 mm to approximately 2 mm. In certain example embodiment, each chamber in the base has the same height. In certain example embodiments, a chamber 115 or a group of chambers 115 may have a shorter height relative to another chamber(s) 115 on the device 100. For example, chambers 115 that will be used to conduct imaging assays may be formed with a more shallow height. The reduced chamber volume may be selected to allow full thickness imaging of the 3D cell/tissue culture and compatibility with advanced imaging techniques such as laser confocal, two-photon, and multi-photon microscopy.

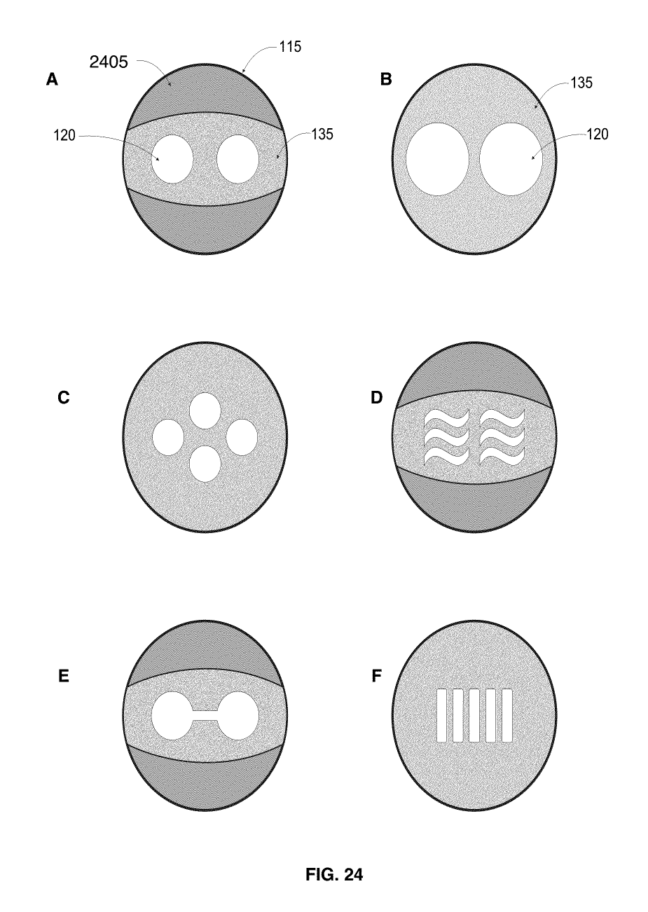

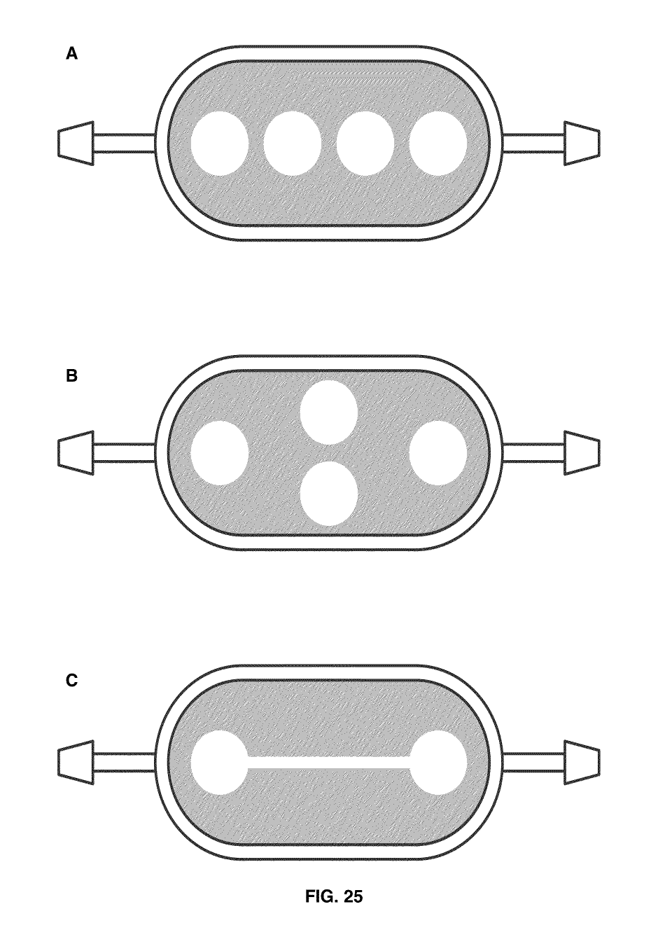

In certain example embodiments, the chamber 115 may be open or void. In certain example embodiments, the chamber 115 may include inserts 135 to form sub-wells 120 within the chamber 115. See FIGS. 1-7. The inserts 135 both define a well space for initial seeding of cells and placement of a three-dimensional cell anchorage construct (3D) for the cells to grow into, upon, or within. The sub-wells 120 defined by the inserts 135 may be round, square, oval, rectangular, diamond, or oblong individual shapes. See FIG. 24. The sub-wells 120 facilitate culture of one or more cell types. For example, the insert may facilitate mixed co-culture or segregated co-culture of multiple cell types. Multiple sub-wells and/or patterns within an insert may be used to facilitate culture of multiple cell types, sampling regions for media aliquots, analytical regions for spectrometry, colorimetric, fluorimetric, and bioluminescent readings, or for specific assay configurations, such as angiogenesis assays, cell migrations assays, metastasis and invasion assays. An insert may define a single sub-well 120 or multiple sub-wells 120 in various arrangements. Example sub-well configurations are shown in FIGS. 24 and 25. The inserts may further define a bridge connecting two or more sub-wells. See FIG. 24E and FIG. 25C. Such a bridge arrangement may be used to conduct various cell migration assays, such as an angiogenesis assay. In certain example embodiments, the bridge space may be filled with a synthetic or natural protein hydrogel material. In certain example embodiments, multiple sub-wells 120 may be arranged is a particular shape, such as a square, oval, diamond, star, or any other geometric shape. In certain example embodiments, one or more sub-wells 120 may be divided in to separate areas. For example, a circular sub-well 120 may be divided in to one or more parts.

The insert 135 may be constructed of and/or include a variety of materials. For example, the inserts 135 may be constructed from a solid material or a porous material. The inserts 135 may be made with materials or coated to prevent cell attachment. The inserts 135 may be clear and transparent to allow visualization of the cell culture from the side. The inserts may be opaque black to prevent light scatter for improved colorimetric absorbance and fluorimetric readings. The inserts may be opaque white to provide for improved bioluminescent readings.

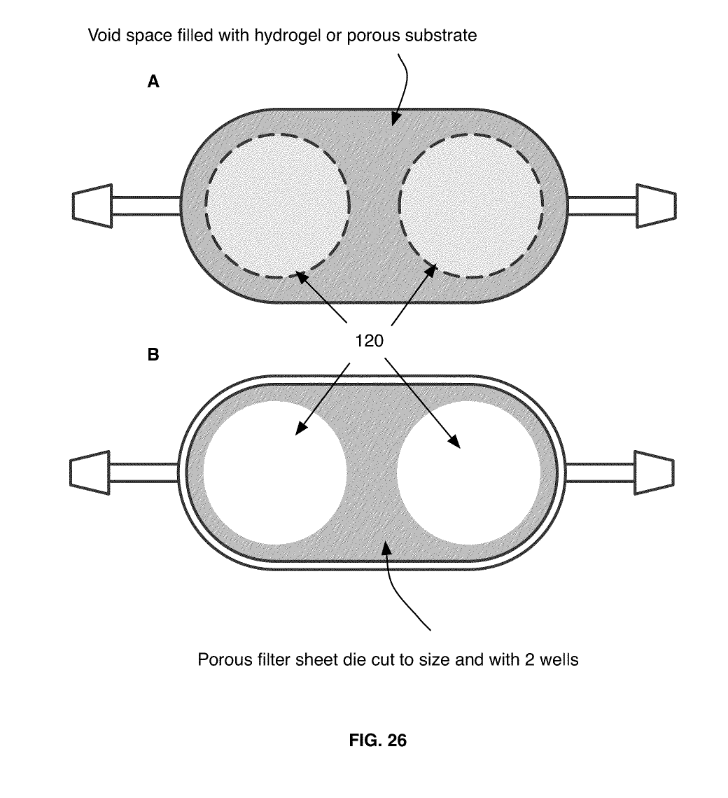

In certain example embodiments, the insert may be porous insert. For example, the porous insert may be a bioinert porous foam, a hydrogel, sintered particles, or stacked fibers. Porous insert materials may include die cut or molded porous filter material. The porous insert material may be cut at an oversized height so that it can be compressed into place to effectively seal against the lid and/or imaging window of the chamber. The porous insert material may be tube shaped. In certain example embodiments, two tubes of different pore size may be used to create a well wall of varying porosity. In certain example embodiments, a porous filter of sufficient thickness may be die cut to the footprint of the incubation chamber to define sub-wells within the incubation chamber 115. See FIG. 26.

As shown in FIG. 26 the void space between sub-wells and the chamber wall may be filled with a substrate. The substrate may be a hydrogel, a porous scaffold, a porous filter, or a combination thereof. The substrate may help position and hold the well forming inserts in place. The pore size of the substrate may be same or different pore size from the pore size of the insert wall. In certain example embodiments, the substrate may be a non-porous substrate. Hydrogel substrates may used for angiogenesis, migration, metastasis, and invasion assays as well as cell proliferation and expansion. That is, the void space may be filled with hydrogel or porous scaffold material that, in certain example embodiments, allows cell growth into the intermediary hydrogel and/or to ensure dispersed perfusion around the core of a cell-scaffold construct. The hydrogel insert may also be used to house an additional cell population. The additional cell population may be the same cell population as that in the sub-wells, or a different cell population from that in the sub-wells.

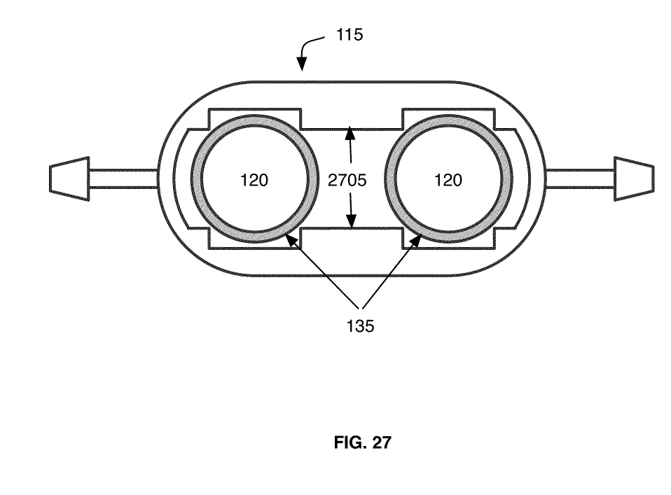

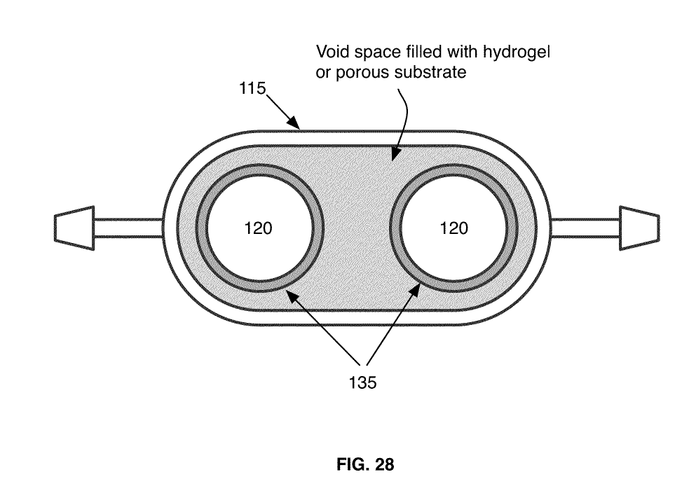

Referring to FIG. 27, in accordance with certain example embodiments, the chamber 115 may include positioning blocks 2705 molded as part of the chamber 115 that hold porous inserts 135 in place. In certain example embodiments, the positioning blocks may be spaced to hold multiple inserts 135 to form sub-wells 120 of diameter similar to 12, 24, 48, 96 or 384 well plates. Alternatively, the inserts 135 may be held in place by filling the void space in the chamber with a porous substrate or hydrogel. See FIG. 28.

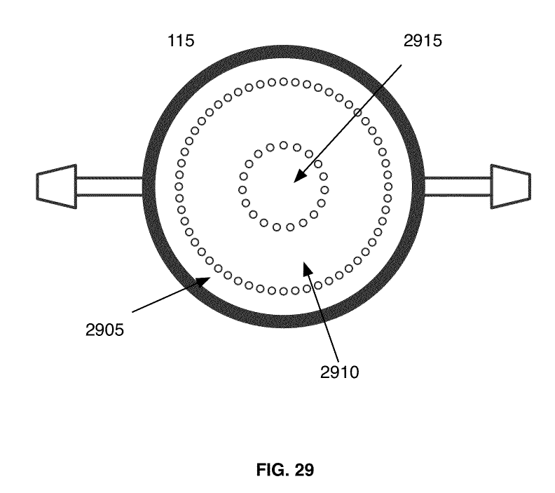

In certain example embodiments, the shape of one or more sub-wells formed by an insert may include a tube-shaped sub-well, such as a sub-well with a hollow, cylindrical shape. In certain example embodiments, the one or more sub-wells may include an inner, pillar-type ring well (or inner pillar well). Additionally or alternatively, one or more sub-wells may include a pillar ring within pillar-ring-type sub-well. As shown in FIG. 29 for example, the pillar ring within pillar-ring-type sub-well may include an outer media well 2905, a cell-scaffold region 2910, and a central plate reader well 2915. In certain example embodiments, the pillar ring within pillar-ring-type sub-well may include multiple sub-well regions that are arranged in a concentric or nested-type pattern.

In certain example embodiments, the portions of a sub-well may be different shapes, such as rectangles, squares, diamonds, or other shapes. For example, the sub-well may include a nested-type series of squares. In certain example embodiments, a single sub-well may include different shapes. For example, a circular sub-well may contain a square, and the square may contain a circle or other shape nested therein.

The layout of the culture area and associated sub-well 120 wall can be adjusted in both shape and size to, for example, reduce the distance proximity that the majority of cells have to the wall. Such adjustments may be useful, for example, when cells are cultured in a 3D cell anchorage and there is a need for higher levels of soluble factors produced by the cells to be introduced into the environment. For example, the shape may be circular versus linear, rectangular, or circular versus multiple, smaller circle-shaped sub-wells, allowing for adjustment of the surface area to volume ratio for the 3D cell construct.

In certain example embodiments, the sub-well may contain a cell scaffold with the insert ensuring dispersed perfusion to and around the cell-scaffold construct. The term “cell scaffold” as used herein refers to one or more articles upon which cells can attach and develop. For instance, the term “cell scaffold” can refer to a single continuous scaffold, multiple discrete 3D scaffolds, or a combination thereof. The terms “cell scaffold,” “cellular scaffold,” and “scaffold” are intended to be synonymous. Any suitable cell scaffold as is generally known in the art can be located in the culture chamber to provide anchorage sites for cells and to encourage the development of a three-dimensional cellular construct within the culture chamber. For purposes of the present disclosure, the term “continuous scaffold” is herein defined to refer to a construct suitable for use as a cellular scaffold that can be utilized alone as a single, three-dimensional entity. A continuous scaffold is usually porous in nature and has a semi-fixed shape. Continuous scaffolds are well known in the art and can be formed of many materials, e.g., coral, collagen, calcium phosphates, synthetic polymers, natural polymers and the like, and are usually pre-formed to a specific shape designed for the location in which they will be placed. Continuous scaffolds are usually seeded with the desired cells through absorption and cellular migration, often coupled with application of pressure through simple stirring, pulsatile perfusion methods or application of centrifugal force. Discrete scaffolds are smaller entities, such as beads, rods, tubes, fragments, or the like. When utilized as a cellular anchorage, a plurality of identical or a mixture of different discrete scaffolds can be loaded with cells and/or other agents and located within a void where the plurality of entities can function as a single cellular anchorage device. Exemplary discrete scaffolds suitable for use in the present invention that have been found particularly suitable for use in vivo are described further in U.S. Pat. No. 6,991,652 to Burg, which is incorporated herein by reference. A cellular scaffold formed of a plurality of discrete scaffolds can be preferred in certain embodiments of the present invention as discrete scaffolds can facilitate uniform cell distribution throughout the anchorage and can also allow good flow characteristics throughout the anchorage as well as encouraging the development of a three-dimensional cellular construct.

In one example embodiment, for instance when considering a cellular scaffold including multiple discrete scaffolds, the scaffold can be seeded with cells following assembly and sterilization of the system. For example, an embodiment including multiple discrete scaffolds can be seeded in one operation or several sequential operations. Optionally, the anchorage can be pre-seeded, prior to assembly of the system. In one embodiment, the scaffold can include a combination of both pre-seeded discrete scaffolds and discrete scaffolds that have not been seeded with cells prior to assembly of the system. The good flow characteristics possible throughout a plurality of discrete scaffolds can also provide for good transport of nutrients to and waste from the developing cells, and thus can encourage not only healthy growth and development of the individual cells throughout the scaffold, but can also encourage development of a unified three-dimensional cellular construct within the culture chamber.

The materials that are used in forming an scaffold can generally be any suitable biocompatible material. In one embodiment, the materials forming a cellular scaffold can be biodegradable. For instance, a cellular scaffold can include biodegradable synthetic polymeric scaffold materials such as, for example, polylactide, chondroitin sulfate (a proteoglycan component), polyesters, polyethylene glycols, polycarbonates, polyvinyl alcohols, polyacrylamides, polyamides, polyacrylates, polyesters, polyetheresters, polymethacrylates, polyurethanes, polycaprolactone, polyphophazenes, polyorthoesters, polyglycolide, copolymers of lysine and lactic acid, copolymers of lysine-RGD and lactic acid, and the like, and copolymers of the same. Optionally, an anchorage can include naturally derived biodegradable materials including, but not limited to chitosan, agarose, alginate, collagen, hyaluronic acid, and carrageenan (a carboxylated seaweed polysaccharide), demineralized bone matrix, and the like, and copolymers of the same.

A biodegradable scaffold can include factors that can be released as the scaffold(s) degrade. For example, an anchorage can include within or on a scaffold one or more factors that can trigger cellular events. According to this embodiment, as the scaffold(s) forming the cellular anchorage degrades, the factors can be released to interact with the cells.

### Adapter for Microwell Plate

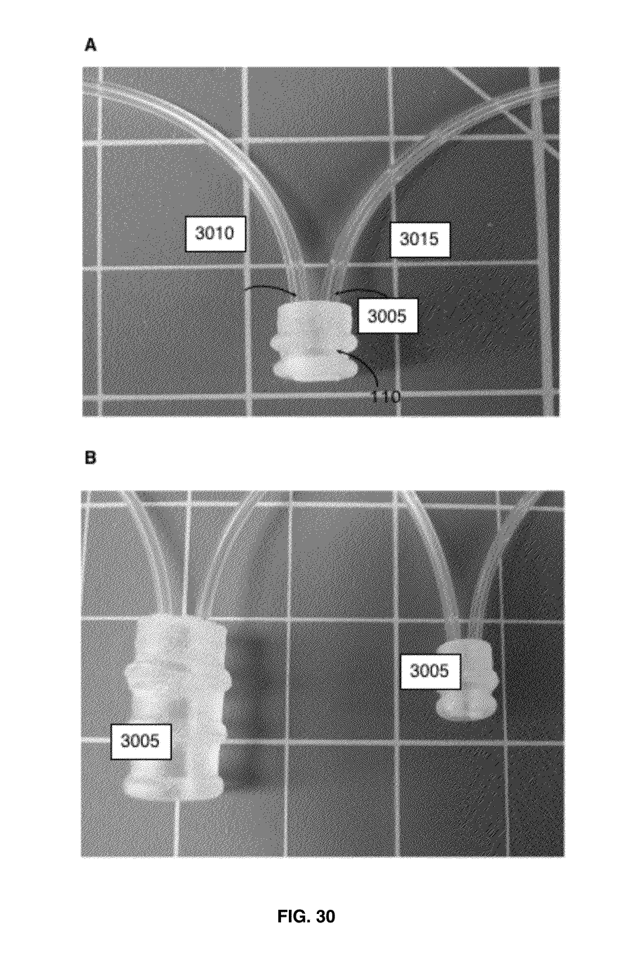

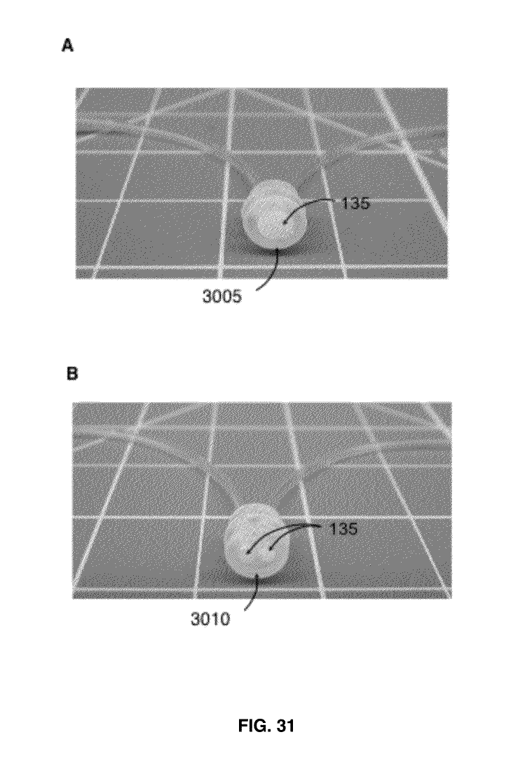

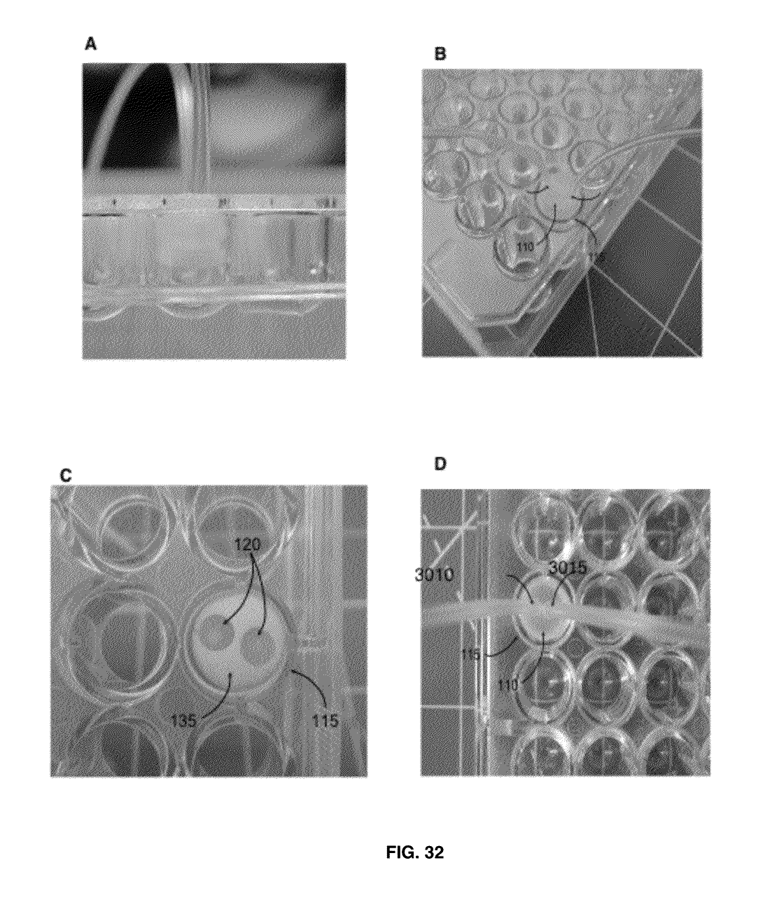

In another aspect, embodiments disclosed herein provide an adapter for converting existing individual microwell plate wells into perfused three-dimensional incubation chambers. As shown in FIGS. 30-32, in accordance with certain example embodiments, the adapter comprises a lid 3005, the lid 3005 defining an inlet channel 3010 and an outlet channel 3015. The lid further comprises an insert 135 as described above. For example, the insert 135 may define the bottom portion of the lid 3005. The inlet and outlet channel 3010 connect to tubing, the tubing used to connect the adapter to a pump source or to another adapter. In certain example embodiments, the insert may be sized to fit within a standard microplate well (such as a 12, 24, 48, 96 or 384 sized well); and define a sub-well as described herein. In certain example embodiments, the insert 135 comprises a non-porous region surrounding a porous region, the porous region surrounding one or more sub-wells 120 defined by the insert 135. The insert may be selected to prevent drug absorption or binding of biological protein therapeutics.

### Hard Top Layer, Soft Bottom Layer Devices

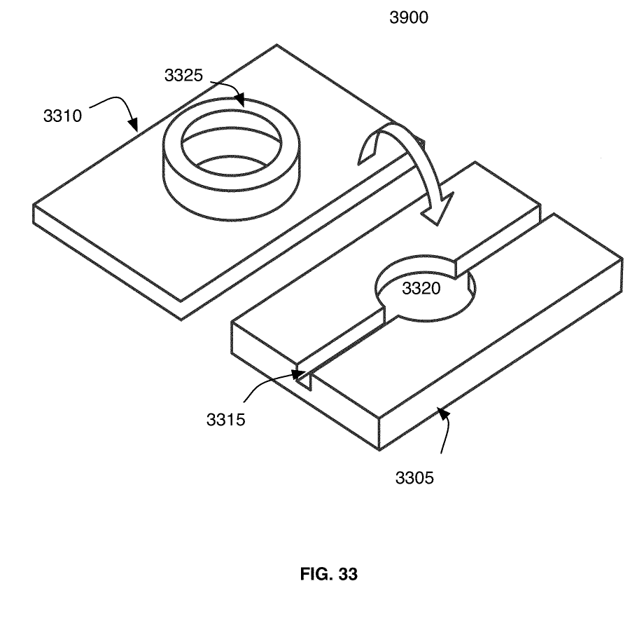

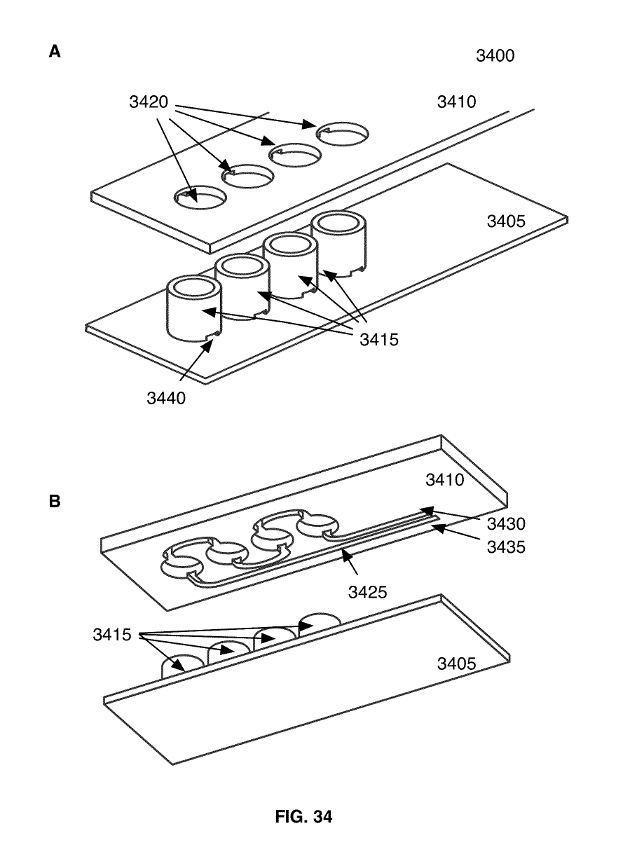

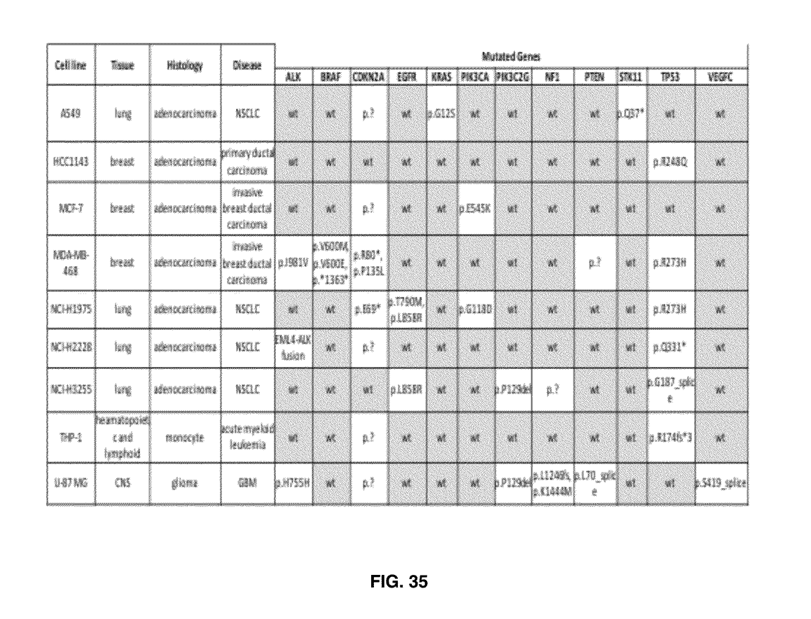

In another aspect, bioreactor devices comprising an elastomeric bottom layer defining one or more fluid channels and one or more culture chambers sealed to a hard top layer are disclosed. Referring to FIG. 33, in accordance with certain example embodiments, the bioreactor device 3900 comprises a soft elastomeric layer 3305. The soft elastomeric layer 3305 defines one or more fluid channels 3315 and one or more holes that completely traverse the soft layer 3320. The soft elastomeric layer 3305 may further comprise a channel compression interface, allowing a pump to interface from the bottom of the device and move up to engage the chip. The device 3300 further comprises a hard top layer 3310, wherein the hard layer 3310 defines a chamber access port 3325 with channels through the wall of the chamber access port at its base (not shown). The chamber access port 3325 aligns with traversing hole 3320 allowing fluid access to the microfluidic channels through the channels in the base of the chamber access port 3325. Including the chamber access port 3325 in the top hard layer reduces manufacturing steps as there is no need to drill inlets in the soft elastomeric layer as done using existing techniques. In addition, the chamber access port 3325 could be shaped as a connection, for example a luer lock, providing an alternative way to connect the device 3300 to a media reservoir that does not require inserting tubing into inlets drilled in the soft elastomeric layer 3305.

In certain example embodiments, the chamber access port 3325 is assembled by rotating the top rigid layer 3310 as indicated in FIG. 33 such that the chamber access port 3325 is inserted through the hole 3320 in the soft layer 3305 such that the open end of the chamber access port 3325 is face down and the channels in the wall of the chamber access port 3325 align with the one or more fluid channels 3315 of the soft layer 3305. In certain example embodiments, the chamber access port 3325 may be formed to create an open-ended cylindrical chamber useful for cell culture such that when the assembled device is inverted (i.e. the open end of the cylindrical chamber faces up). Such an embodiment would function similar to a well in a microplate allowing access to the chamber by pipette or enabling the insertion and extraction of cell scaffolding materials, which is not typically available using existing microfluidic culture architectures. In addition, such an open-ended cylinder made of the hard layer 3310 would be easier to temporarily close and seal off with a cap or lid then one made of a soft layer material. All imaging may be done through the top hard layer 3310 as there is no soft elastomeric layer barrier.

In certain example embodiments, the chamber access port 3325 may have one or more channels in the walls of the chamber access port 3325 that enter into the chamber at different heights to create strategic flow patterns or gradients that result in flow throughout the cylinder. For example, one high and one low channel entry point may be created on opposite sides of the chamber access port 3325. Alternatively, two channel entry points may be created on opposite sides of the chamber access port to allow flow only in the bottom region.

Example soft and hard materials for use in each layer, and chamber and fluid circuit volumes discussed in detail above further apply to this embodiment. In addition, the inserts 135 described above may also be used with this embodiment. The embodiment shown in FIG. 33 depicts a single chamber with inlet and outlet channels shown in the soft layer. However, multi-chamber configurations are contemplated. Each chamber in a multi-chamber configuration may have its own inlet and outlet channel, or may be connected in series to other chambers via the inlet and outlet channels.

### Hard Bottom Layer, Soft Top Layer Devices

In another aspect, bioreactor devices comprising a hard material bottom layer 3405 and a soft elastomeric top layer 3410 are disclosed. The hard bottom layer 3405 defines one or more chambers 3415. The top soft elastomeric top layer 3410 comprises openings 3420 for receiving the one or more chambers 3415 and allowing the soft top layer to interface with the hard bottom layer 3405 at the bottom of the chamber 3415. The soft top elastomeric layer 3410 further defines a fluid circuit 3425 comprising an inlet 3430 and an outlet 3435 and connecting the chambers 3415 to one another along the fluid circuit 3425. The fluid circuit 3425 is example and other circuits and arrangements of the chambers 3415 is contemplated. The chambers 3415 have a set of openings 3440 in the wall of the chamber that align with the fluid circuit 3425 when the soft elastomeric top layer 3410 is seated on the bottom hard layer 3405. The inlet could be created as a feature of the hard layer 3405 reducing manufacturing steps since there is no need to drill inlets in soft elastomeric layer. The chambers 3415 may be shaped as a luer locks or other connectors providing an alternative way to connect the device 3400 to a media reservoir that does not require inserting tubing into inlets drilled in the soft elastomeric layer. Such an embodiment would function similar to a well in a multi-well plate allowing access to the chamber by pipette or enabling the insertion and extraction of cell scaffolding materials, which is not typically available using existing microfluidic culture architectures. In addition, such an open-ended cylinder made of the hard layer material would be easier to temporarily close and seal off with a cap or lid then one made of a soft layer material.

Example soft and hard materials for use in each layer, and chamber and fluid circuit volumes discussed in detail above apply further apply to this embodiment. In addition, the inserts 135 described above may also be used with this embodiment.

### Methods of Use

The following methods are by way of example only. The devices disclosed herein may be used in any method that requires or could benefit from the ability to grow and develop in a three-dimensional aspect, and require or benefit from the use of small culture volumes that allow for direct analytics to be done on or by the device, allow full thickness imaging, and permit continuous monitoring and measurement of the growth of a cell culture. The systems disclosed herein through the various chamber arrangements, use of integrated fluid circuits, ability to adapt existing microwell plate wells, and various well formations through the use of inserts provide a high degree of flexibility and scalability for a wide array of clinical and research uses.

In one example embodiment, a method of culturing on or more populations of cells in a three-dimensional culture comprises culturing one or more cell populations in a bioreactor device disclosed herein. A single population may be grown in a single chamber. Multiple cell populations may be grown in multiple chambers. The multiple cell populations may be of the same cell type or of different cell types. In certain example embodiments, the multiple cell populations comprise 2-30 different cell populations. In one example embodiment, the multiple cell populations comprise 2, 3, 4, 5, 6, 7, 8, 9, or 10 different cell populations. In certain example embodiments multiple different populations can be grown in different sub-wells within the same chamber. In certain example embodiments, the cell population is a in vitro cell population derived from an existing cell line. In certain other example embodiments, the cell population is derived directly form a biological sample taken from a subject. In certain example embodiments, the biological sample is a biopsy sample. In certain example embodiments the biopsy sample is from a cancer patient. The cancer may be lung cancer, breast cancer, ovarian cancer, pancreatic cancer, or glioblastoma. In certain example embodiments, the biological sample is a clinical isolate infected with a pathogen such as a bacteria, fungus, or virus. In certain example embodiments, the cell population may be derived by initially culturing a single cell.

In another example embodiment, a method of culturing one or more cell populations for ex vivo implantation comprises culturing one or more cell populations in a bioreactor device disclosed herein. In certain other example embodiments, the cell population is an ex vivo derived cell population. In certain example embodiments, the ex vivo derived cell population sample is a biopsy sample from a particular individual. In certain example embodiments, the ex vivo derived cell population includes undifferentiated stem cells. A single population may be grown in a single chamber. Multiple cell populations may be grown in multiple chambers. The multiple cell populations may be of the same cell type or of different cell types. In certain example embodiments, the culture conditions created by the disclosed device result in desirable stem cell differentiation and the creation of isolated, differentiated cells for implantation. In other embodiments, a similar process is utilized to alter the original cells, e.g. by transfection or other means, prior to their implantation. In other embodiments, a similar process is used to increase their cell number without impeding changes to their phenotype prior to their implantation

In another example embodiment, a method of assessing responsiveness to therapeutic agents comprises culturing one or more populations of cells in a bioreactor device disclosed herein. The one or more cell populations are then cultured in the presence of a therapeutic agent for a defined period of time. After the defined period of time the one or more cell populations are screened for responsiveness to the therapeutic agent. For example, a change in cell phenotype may be observed using microscopy techniques, cell cytotoxicity may be assessed using an appropriate biomarker, such as LDH, gene expression for one or more relevant biomarkers may be assessed though lysis and nucleic acid amplification and/or sequencing or by in situ hybridization techniques such as FISH, one or more protein biomarkers may be detected using immunoassays or immunostaining techniques, and/or the cell culture media may be assessed for the presence of various biomarkers of cell metabolism or other cell functions. The appropriate screen for pharmacokinetic effect will depend on the class of therapeutic agent screened and the corresponding cell pathway targeted. In certain example embodiments, a panel of pharmacokinetic effects may be tested and involve techniques that do and not require cell lysis or fixing of cells. Accordingly, in some example embodiments, a cell population may be grown in the sub-wells of a first chamber and used to screen for a pharmacokinetic effect using a technique that requires cell lysis or fixation, while the same cell population may be grown in the sub-wells of a second chamber and used to screen for a pharmacokinetic effect using a technique that does not require cell lysis or fixation. In certain example embodiments, multiple samples of the same cell population may be cultured in different chambers to allow for assessment of pharmacokinetic effect at different time points, especially where the technique used to assess the pharmacokinetic effect requires cell lysis or fixation. The change in pharmacokinetic effect may be determined in reference to a control population not exposed to the one or more therapeutic agents.

In certain example embodiments, a method for selecting the responsiveness of a subject to therapeutic agents ex vivo comprises culturing one or more cells derived from a biopsy sample of a subject in the sub-wells of multiple chambers of a bioreactor device disclosed herein, exposing the cultured one or more cells candidate therapeutic agents, and/or exposing each chamber to a different concentration of the candidate therapeutic agent, and measuring the responsiveness of the one or more cells to each candidate therapeutic agent and/or concentration, and selecting a therapeutic agent and/or dosage based on the observed responsiveness. The technique used to measure responsiveness will depend on the disease type and class of therapeutic agent. For example, for a therapeutic agent designed to restore particular cell function, responsiveness may be measured by a change in appropriate biomarker level of cell morphological change. For a therapeutic agent designed to kill or inhibit the one or more cells, responsiveness may be measured by biomarkers for cytotoxicity and/or morphological changes. In certain example embodiments, the biopsy sample is a cancer biopsy sample. In certain example embodiments, the cancer is breast cancer, ovarian cancer, lung cancer, pancreatic cancer, or glioblastoma.

In certain example embodiments, the cells derived from a biopsy sample are grown in a microtumor environment comprising one or more additional non-cancer cell types. In certain example embodiments, the one or more additional non-cancer cell types comprise epithelial cells, fibroblasts, adipocytes, endothelial cells, tumor associate macrophages, T-cells, B-cells or a combination thereof.

In certain example embodiments, a method for screening the effects of genetic perturbations on cell biology comprises culturing a plurality of cell population samples in the sub-wells of a plurality of chambers. Each sample cell population may then receive a separate genetic perturbation. Genetic permutations may include deletions, insertions, substitutions, or epigenetic changes. For example, target genes may be knocked down using RNAi techniques. Alternatively target genes may be knocked down or target sequences modified using, for example, zinc-finger nucleases (ZFNs), transcription activator-like effector nucleases (TALENs) or CRISPR/Cas-based methods. The resulting effects on cell expression and/or phenotype may then be detected using known techniques.

In another example embodiments, it is contemplated the bioreactor systems disclosed herein can be utilized for the replication of biological conditions such as cell migration/invasion such as, for example and without limitation, wound healing, metastasis, vasculogenesis, immune responses, angiogenesis, tumor formation, and chemotaxis. Additionally the bioreactor systems disclosed herein can be utilized for the replication of biological conditions involved in cellular proliferation, cell survival and attachment. Used in such a manner the bioreactor device provides the extracellular contact and milieu to more closely replicate an intact biological system. The cell may be cultured in media containing factors that encourage cell growth and/or migration. In certain example embodiments, inserts defining two sub-wells and a channel between the two sub-wells are used to assess cell migration and invasion. See FIG. 25C. Migratory cells move across the channel from the first well to the second well while non-migratory cells remain in the first well.

In another example embodiment, undifferentiated stem cells can be located in a first well or chamber of the bioreactor devices, and one or more types of feeder cells can be located in an adjacent sub-well or chamber, which, as one skilled in the art will appreciate, can be in selective communication with the first well or chamber. Such a method can be used to, for example and without limitation, study the triggering mechanisms involved in stem cell differentiation or to provide isolated differentiated cells for implantation.

According to the methods disclosed herein, cells can be grown in an environment that comprises the biochemical products of different cells types, at least some of which may be necessary for growth, differentiation, or development of the desired cells. However, it is contemplated that different cell types can be maintained in a physically isolated state during their grow and development using the inserts and/or chambers disclosed herein while allowing fluid communication between the different cell populations, for example via the fluid circuits.

Cells and tissues used in the bioreactor devices and methods disclosed herein can be obtained by any method known to those skilled in the art. Examples of source cells and tissues include without limitation purchase from a reliable vendor, blood—including peripheral blood and peripheral blood mononuclear cells), tissue biopsy samples, and specimens acquired by pulmonary lavage. The source of cells and tissues obtained from blood, biopsy, or other ex vivo means can be any subject having tissue or cells with the desired characteristics including subjects with abnormal cells or tissues which are characteristic of a disease condition such as cancer. It is also understood that there may be times where one of skill in the art desires normal tissues or cells. For example, responsiveness or other cellular changes in a test population of cells may be measured against a control or normal population of cells that are not exposed to the experimental conditions. Thus, also disclosed herein are tissues and cells obtained from a normal subject, wherein ‘normal’ refers to any subject not suffering from a disease or condition that affects the cells or tissues being obtained.

### EXAMPLES

One of the most complex 3D bioengineered models of breast cancer to date is a tri-culture model. These models always include epithelial cells and fibroblasts and vary based upon their incorporation of adipocytes, endothelial cells or immune cells. S. Krause, et al., TISSUE ENG PART C. METHODS, 14 (2008) 261-271. A porous scaffold will be used to create a modular system composed of a fat module and an epithelial module that also contains fibroblasts. This modular system has been demonstrated to facilitate 1) the differentiation of adipose-derived stem cells into a separate fatty tissue without the chemicals necessary for differentiation having a negative impact upon the breast cancer epithelial cells and 2) the development of tri-culture breast cancer tissue with patient derived tumor cells that became available with little notice.

There are numerous benefits to utilizing a porous scaffolding system as they: 1) are biocompatible and many are FDA approved biomaterial, 2) have been utilized to sustain tissue structures for up to months/years, 3) are compatible with vascular systems, 4) can withstand the mechanical stresses of perfusion flow, 5) have been utilized for multiple tissue types, and 6) are adaptable for optical imaging systems. G. H. Altman et al. BIOMATERIALS, 24 (2003) 401-416; P. Gomez, III et al. BIOMATERIALS, 32 (2011) 7562-7570; M. House et al. TISSUE ENG PART A, 16 (2010) 2101-2112; H. J. Kim et al. BONE, 42 (2008) 1226-1234; M. Lovett et al. BIOMATERIALS, 28 (2007) 5271-5279; J. R. Mauney et al. BIOMATERIALS, 28 (2007) 5280-5290; E. M. Pritchard and D. L. Kaplan, EXPERT. OPIN. DRUG DELIV., 8 (2011) 797-811; C. Vepari and D. L. Kaplan, PROG. POLYM. SCI., 32 (2007) 991-1007. The use of porous scaffolds as sponges infused with ECM and different cells types forms a modular tissue unit that may be easily moved and combined with other modular units to form controllable complex tissues.

The in vivo microenvironment of the breast consists of epithelial cells, fibroblasts, adipocytes, and endothelial cells in contact with extracellular matrix proteins (ECM). McCave et al. J. MAMMARY. GLAND. BIOL. NEOPLASIA., 15 (2010) 291-299. The complexity of the created ex vivo microenvironment will be increased relative to previous tri-culture models by adding in both endothelial cells and an immune component cells in the form of monocytes/macrophages. The porous scaffold infused with cells in a mixture of Matrigel and Collagen I will be used as the ECM components as they have been shown to promote the formation of terminal ductal lobular units (TDLUs) and acini while maintaining the cells in an environment where they remain healthy.

Previous tri-culture models will be further adapted to a tetra-culture model by utilizing the same cells as the tri-culture model, including human mammary epithelial cells (HMEC) for the normal model and MCF7 (ER+) and HCC1143 (TNBC) for the cancer model, human mammary fibroblasts (HMF) and human adipose-derived stem cells (ASC) with the addition of human umbilical vein endothelial cells (HUVEC) to form the tetra-culture. The HUVECs will be utilized for initial short-term testing, but will then be replaced by human mammary microvascular endothelial cells (HMMEC) so as to keep the system mammary specific. HUVEC/HMMEC will be seeded onto a porous scaffold which is precut to specific dimensions (5 mm diameter by 2.5 mm height) and allowed to grow for 1 week after which ASCs will be added to the scaffold. The scaffolds will be incubated in a mixture of ASC differentiation media and endothelial growth media for 1 week after which they will be cultured in a mixture of ASC growth media and endothelial growth media. The epithelial module will be formed by mixing HMEC, MCF7, or HCC1143 with HMF in a mixture of Matrigel to Collagen I. This mixture of cells and ECM will then be infused into a porous scaffold which is precut to specific dimensions (5 mm diameter by 2.5 mm height). These tissue disks will then be loaded into the sub-wells of the bioreactor devices disclosed herein. After loading the tissue disks into the sub-wells of the bioreactor devices, the devices will be assembled and connected to a perfusion circuit containing cell-specific medium. Gas exchange is enabled in the closed-loop perfusion system via a proprietary PET membrane, and media is dynamically perfused via a programmable syringe pump configuration (KD Scientific) with a dynamic medium perfusion flow of 1-50 uL/min for the length of the experiment. All bioreactor devices will be maintained under standard cell culture conditions (i.e. 37° C. humidified 5% CO2/95% air environment).

To test the tetra-culture medium and ensure that the endothelial growth media is compatible with all four cell types, each individual cell type will first be tested in 2D culture in a specified ratio or ratios of all four cell specific mediums over 7 days. The combo medium's effects upon cell viability (measured by PrestoBlue) and cell number (dsDNA measured via PicoGreen) will be measured with cell specific growth medium utilized as a control. A short-term 3D static experiment will also be performed to ensure proper tissue morphology which will consist of the formation of TDLU/acini in the normal model and endothelial cell branching/tubule formation (CD31+). Endothelial cells and ASCs will also be cultured in 2D in a mixture of endothelial growth medium and ASC differentiation medium over 7 days and monitored for cell viability and cell proliferation as compared to cell specific growth medium. ASC differentiation will also be monitored by oil red O staining.

Long-term perfusion experiments (4 weeks) will be performed with static correlates for both the normal model (HMECs) and the cancer model (MCF7). Analytical endpoints will be taken at weekly intervals for all lytic assays and shorter intervals for non-lytic perfusate analysis. Media will be changed periodically. The formation of TDLUs/acini and endothelial cell branching/tubule formation as determined by brightfield microscopy and H&E, production of caseins by RT-PCR, replication of the secreted biomarker profile and expression of cell specific markers (E-cadherin, CD31, leptin, adiponectin, α-sma, oil red O) by RT-PCR and immunofluorescence at weeks 2 and 4 will determine successful stromal culture conditions.

Angiogenesis will be tested in the tissue model by stimulating the cultures with varying concentrations of VEGF and counting the number of sprouts and the length of sprouts compared to untreated controls in both static and perfusion cultures. VEGF will be applied and tubules will be visualized using confocal microscopy by staining with PECAM-1. HUVECs/HMMECs alone in Matrigel will be used as controls. A comparison between the tri- and tetra-culture models in terms of gene expression and both epigenetic and miRNA regulated gene expression will be conducted. This data may be utilized for comparison to patient generated data. To perform this comparison, whole genome cDNA microarray data will be generated as well as epigenetic and miRNA microarray data in-house.

Triple-negative breast cancers are resistant to current targeted therapies but may see some benefit from anti-angiogenesis compounds. Bevacizumab will be used in combination with paclitaxel (as utilized in the clinic) as a baseline test on the microtumor model to see if it responds similar to clinical trial data. For this study, angiogenesis will be induced through VEGF addition and then a dose curve centered at the IC50 for each drug will be generated. The endothelial cells will be pre-labeled. Cellular cytotoxicity will be measured by LDH release into the supernatant, vascularization will be monitored by confocal microscopy of the labeled endothelial cells. Length and number of sprouts will be counted and related to bevacizumab dosage. Untreated cultures and the tri- culture model will be used as a control since bevacizumab cannot function in this tissue by affecting angiogenesis. The effectiveness of this model for modeling responses to traditional treatments (tamoxifen, cisplatin) will also be measured and the IC50s will be compared to static culture and data previously generated for the tri-culture model.

Tumor associated macrophages (TAMs) in the perfused heterotypic 3D breast microtumor will be incorporated and biologic read outs of macrophage invasion/infiltration and pro-cancer cytokine/chemokine profile to assess changes in phenotype over time in culture will be correlated. This will be achieved by analyzing the immunophenotype of perfused THP-1 or differentiated PBMCs obtained from discarded blood or through commercial suppliers. Multiplex bead based immunoassays (Miltenyi) will be used to determine M1 versus M2. For invasion, myeloid-derived cells will be pre-labeled and infiltration over time will be monitored qualitatively with multi-photon microscopy (MPM). Pro-tumorigenic growth and promotion of endothelial structures will be compared with 3D microtumors without TAMs via immuno-histochemistry (IHC). MPM will be used to monitor the effect of macrophages on promotion of vascular structures (through the secretion of proangiogenic factors). J. D. Lathia et al. PLoS ONE, 6 (2011) e24807. If a pro-tumorigenic effect of macrophages is demonstrated within the system, ibrutinib, dexamethasone, and anti-csf-1 antibodies will be applied to reverse the TAM mediated effects.

### Example 2

### Patient-Derived 3D Tetra-Culture Breast Cancer Microtumors

Patient-derived 3D tetra-culture breast cancer microtumors will be established by replacing the MCF7 cells with tumor cells isolated from breast cancer patients. Human tumor tissue will arrive in the lab after removal from the patient at which point cellular processing will immediately commence. Isolated primary breast cancer cells will be utilized in a number of assays all designed to better inform upon the utility of the personalized 3D microtumor. Cell samples will be utilized to isolate DNA, RNA, miRNA, and protein. The DNA will be used to compare to epigenetic biomarkers established above, RNA will be used to examine basic gene expression (ER, PR, Her2, casein), miRNA will be used to examine miRNA biomarkers in comparison to those determined above, and protein will be used to examine the expression of genes regulated by the epigenetic/miRNA biomarkers. Once the 3D breast model has been established with primary human epithelial and stromal breast cancer cells, a panel of patient derived 3D breast microtumors will be tested against a panel of combination therapies. In order to compare with existing databases next generation sequencing (NGS) will be performed, and molecular matched breast cancer cell lines will be compared with responses in 2D and simple 3D matrigel cultures. Normalized responses will be depicted as IC50s using 4 parameter linear regression.

A key component of the validation study is to compare genotypic and phenotypic drift, as well as therapy response of the 3D microtumors with patient-derived xenografts developed from matched patient samples. This process will occur with the initial tumor biopsies in order to provide sufficient PDX models for statistical considerations. Given the difficulty for developing ER+PDXs, preference will be focused on TNBC PDX.

### Example 3

### 3D Glioblastoma Microtumor Model

Multiple investigators have demonstrated that the current state of the art for modeling GBMs is utilizing primary human tissue and initial processing for both in vitro and in vivo investigation. J. D. Lathia et al. PLoS ONE, 6 (2011) e24807; C. Richichi et al. NEOPLASIA., 15 (2013) 840-847; S. J. Smith et al. PLoS ONE, 7 (2012) e52335; D. W. Infanger et al. CANCER RES., 73 (2013) 7079-7089; S. E. Yost et al. PLoS ONE, 8 (2013) e56185; D. R. Laks et al. STEM CELLS, 27 (2009) 980-987. Though it will be important to compare drug response in the final iteration of the 3D GBM microtumor with subtype-matched cell lines, including the GBM NCI 60 cell line prescreen GBM cell line SF268, the development work should begin with isolated GSCs from patient samples. GSC spheroids from primary human GBM tumors will be isolated utilizing established protocols Lathia et al.; Richichi et al; Smith et al.; Infanger et al.; Yost et al. Laks et al; A. B. Hjelmeland et al. CELL DEATH. DIFFER., 18 (2011) 829-840. After informed consent is obtained, surgical specimens not used for diagnostic purposes will be obtained and de-identified followed tumor processing in a dedicated primary culture hood. Tissue fragments will be separated for histologic and molecular characterization. The tissue will be processed and a subset of the cell population will then be assessed for neurosphere culture and expanded.

Cell seeding post expansion will be initially assessed in various scaffolds and ECM at different quantities. It is anticipated that the larger cell numbers will develop tighter cell aggregates within the smaller porosity scaffold discs, but that overall viability will not be substantially different in the perfusion system. Given the need to incorporate other cell types, the potential limit on primary GSCs and the need to allow expansion/growth of the co-cultures over time, it is anticipated that the larger porosity scaffold discs with smaller to intermediate numbers of cells will be optimal for long term perfusion. Short term static culture of GBM cell lines will be used to determine optimal media, consisting of either stem cell media or differentiation media. In addition, media pH will be altered with hydrochloric acid and cell viability and angiogenic secretome in 3D will be assessed. Finally, the effect of reduced oxygen conditions (˜1%) on cell growth and degree of hypoxia/necrosis within spheroids will be assessed.

Analytical techniques will be a combination of non-lytic and lytic assessments of viability (resazurin reduction, Presto Blue, Life Technologies; dsDNA quantification, CyQuant, Life Tech; live cell imaging, calcein AM, Life Tech) and cytotoxicity (LDH release, dead cell imaging, Ethidium HomoDimer, Life Tech). Hypoxia will be monitored in situ and non-destructively with a penetrating hypoxia-specific Lox-1 probe and imaged in situ via laser confocal microscopy S. Zhang et al. CANCER RES., 70 (2010) 4490-4498. IHC will be performed on formalin fixed 3D static tissues to assess GSC stem cell markers and GBM differentiation/proliferation markers. Immunophenotype of seeded cells and extracted cells in 3D culture will be assessed for similar markers by flow cytometry, to demonstrate multilineage differentiation. L. F. Pavon et al. FRONT NEUROL., 4 (2014) 214. Soluble angiogenic factors will measured by multiplexed bead based immunoassay.

Short term assessment of viability and hypoxia will be used to identify the best flow rate, which will need to be re-assessed when endothelial cells are added. Perfused 3D cultures of GBM cell lines and GSCs will be performed over 2 weeks in optimized media conditions (stem cell versus differentiation supplements, acidic versus neutral pH, relative hypoxic versus standard incubator), with 4 analytical time points evaluated. Lytic Analysis: IHC will be performed on fixed 3D static tissues using established fixation and imbedding and slides will be immunostained as stated for GSC and differentiation markers. In addition to above IHC for expression markers, the BrdU incorporation assay will be used to better reflect the proliferative cell population over a 48 hour time period in situ, as this assay may better reflect proliferation than traditional IHC with Ki67. M. Witusik-Perkowska et al. J. NEUROONCOL., 102 (2011) 395-407. Methylation changes will be examined over short term and long term culture experiments. The utilization of bioreactor array platform will enable more efficient and cost effective analysis of the GBM methylome. In addition, methylation status of primary samples can be compared directly with the TCGA to correlate sample tissues with that of the TCGA. Non-lytic analysis: As previously discussed, non-destructive analysis will consist of metabolism, cytotoxicity and in situ imaging using multi-photon microscopy to quantify the redox ratio of different cell populations (i.e. higher proliferating cells in outer layer of spheroids will have a lower redox ratio (NAD/FAD) and slower proliferating intermediate and inner layer cells will have a higher redox ratio. Angiogenic factors will be assessed as previously stated.

The two mainstays of GBM therapy are external beam radiation and temozolomide TMZ. The bioreactor devices disclosed herein are perfectly suited for evaluating radiation sensitivity and resistance (dosimetry, tissue penetration, effects of hypoxia, soluble factors of cytotoxicity, etc.). TMZ will be applied to each system across multiple cell lines and multiple GCSs in 3D GBM microtumors over 7 days and normalized IC50s will be compared with molecular signature and methylation status of MGMT promoter and other 3D microtumor-specific methylome. Importantly, basic correlation with patient response (time to progression [TTP], overall survival [OS] and TCGA data will be established. Stratification will be according to patient demographics and GBM subtypes, as previously stated. Ultimately, TMZ therapy is not curative and progression ensues due to resistant cell populations. Thus, post-treatment analysis will consist of evaluation of the genomic, epigenetic and phenotypic changes of TMZ resistant populations.

The established parameters of the 4 week perfusion 3D GBM microtumor with GSC alone will then be incorporated with endothelial cells (EC). The addition of EC will drive expansion of the GSC population, drive GSC differentiation towards tumor heterogeneity, demonstrate tubulogenesis towards hypoxic regions of GSC 3D aggregates and demonstrate effectiveness of anti-angiogenic agents (bevacizumab, axitinib, etc.). In recapitulating the perivascular niche for GSCs, it will be possible to test agents in combination with TMZ and anti-angiogenic agents to overcome treatment-related resistance seen in the clinic. Furthermore, patient-specific endothelial cells will further contribute to mimic the tumor heterogeneity seen in patients.

In additional experiments, GBM-specific endothelial cells will be isolated, which will then be incorporated into the GBM microtumor for testing. Expression level changes can then be correlated with degree of microvascularity obtained in vitro as well as to anti-angiogenic treatment response that will be patient specific. GBM specific Ecs to be used in development and testing of 3D GBM Microtumors will be isolated, minimally characterized, and processed.

Optimized media and conditions (growth factors, pH, hypoxia) will be assessed using simple viability and cord formation on a small percentage of early passage (2-3) and late passage EC (7-8) of the first 2-3 patient samples to determine similar growth and function between early and late passage. Conditionally immortalized HBECs will be used as comparison throughout optimization experiments. Based on the optimal numbers of HBECs, GECs seeding density will be determined for future experiments. Once these parameters are set, scaffolds with HBECs will be perfused in the disclosed bioreactors at the optimized perfusion rate determined above, and similar non-destructive analysis as performed above will be performed over 2 week cultures to determine HBEC compatibility with 3D perfusion. Viability and cytotoxicity analysis will be performed non-destructively and terminal analysis will be performed with confocal microscopy to evaluate tubular morphology and branching. IL-8 secretion will be measured and correlated with static 2D and 3D conditions.

Ratios of direct co-culture seeding of GSCs and GECs will be assessed in 2 week perfusion experiments. Using non-lytic multi-photon microscopy, two parameters at four time points over the two weeks will be monitored: redox ratio of GSCs in close proximity to Ecs and branching morphology of Ecs. In order to do this, Ecs will be pre-labeled with dyes which will mimic in vivo experiments using dextran to label vascular structures. Lathia et al. It is predicted that the redox ratio of GSCs will be increased (slower proliferation) the closer to branching structures, correlating with slower proliferating fraction and consistent with a more undifferentiated phenotype. Confirmation of redox ratio as a proliferation index will be confirmed by BrdU incorporation assay as described above. Once the optimal co-culture ratio is determined, the benefit of patient-matched GSC and GECs will be demonstrated by comparing unmatched tumor cell lines and unmatched Ecs and demonstrated relative decrease in viability of GSCs and relative decreased tube formation and branching structures in unmatched co-cultures.

The perfused co-culture will be extended for 4 weeks to assess the capacity of GSCs to undergo multi-lineage differentiation, using the same analytical parameters above for comparison (3D GSC microtumor). It is expected that matched GECs will enhance growth of GSCs over the 4 week platform while promoting intra-microtumor multi-lineage heterogeneity as assessed by similar analytical parameters used above. The GSCs will promote enhanced differentiation of GECs towards tubule generation and branching phenotypic. It is expected that the total microtissue will contain a significantly more heterogeneous population, as determined by IHC and flow, than when GSCs are cultured alone in 3D perfusion or than cell lines in 3D co-culture.

Anti-angiogenic agents (bevacizumab, sunitinib, axitinib) will then be tested in the perfused system and monitor the effects of the agents solely by in situ, non-destructive methods using multi-photon microscopy (redox ratio of tumor cells, morphology of structures of Ecs), metabolism, cytotoxicity and confirm a relative decrease in more differentiated, faster proliferating cell population than the GSC population (CD133+) using lytic methods. Effects of anti-angiogenic agents will be compared with cell lines and primary GSC and GECs in 2D.

All publications, patents, and patent applications mentioned herein are incorporated by reference to the same extent as if each individual publication, patent or patent application was specifically and individually indicated to be incorporated by reference in its entirety. In the event of there being a difference between definitions set forth in this application and those in documents incorporated herein by reference, the definitions set forth herein control.

Various modifications and variations of the described methods, pharmaceutical compositions, and kits of the disclosure will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A bioreactor device comprising:
one or more chambers; and
a lid, the lid comprising one or more integrated flow circuits defined therein, the flow circuits comprising flow channels that direct fluids into the one or more chambers.

2. The bioreactor device of claim 1, wherein the one or more chambers are wells of a microwell plate.

3. The bioreactor device of claim 1, further comprising, for each chamber, an insert, the insert defining within the chamber one or more sub-wells.

4. The bioreactor device of claim 1, wherein the lid comprises a first layer and a second layer, wherein the first layer comprises inputs for connecting each flow circuit to a pump, and wherein the flow circuit is defined within the first and second layer.

5. The bioreactor device of claim 4, wherein the second layer further comprises, for each chamber, an extension on the bottom surface of the second layer that seals the second layer to the one or more chambers.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The bioreactor device of claim 1, wherein the one or more chambers further comprise an analytical imaging chamber to facilitate microscopy or spectrometry analysis in the chamber.

22. The bioreactor device of claim 1, wherein the fluidic circuit comprises an analytical imaging window.

23. The bioreactor device of claim 3, wherein the inserts define two or more sub-wells.

24. (canceled)

25. (canceled)

26. (canceled)

27. (canceled)

28. (canceled)

29. (canceled)

30. An adapter to convert microwell plates into three-dimensional culture devices comprising a sealing lid to fit within a well of a microwell plate, the sealing lid comprising an input flow channel and an output flow channel, and an insert defining one or more sub-wells within the well of microwell plate.

31. The adapter of claim 30, wherein the insert comprises a non-porous outer region and a porous region around the sub-wells.

32. The adapter of claim 30, wherein the microplate is a 6, 12, 24, 48, 96, or 384 well-sized microplate.

33. A bioreactor device comprising:
a bottom soft layer defining one or more inlet and outlet fluid channels and one or more openings; and
a rigid top layer comprising one or more chamber access ports to align with the one or more openings, the one or more access ports comprising one or more channels in the walls of the access ports.

34. The bioreactor device of claim 33, wherein the device is assembled by sealing the rigid top layer to the bottom soft layer such that the one or access ports are inserted through the one or more openings and the one or more channels in the walls of the access port are aligned with the inlet and outlet fluid channels of the soft layer.

35. The device of claim 33, wherein the access port defines a cylindrical cell culture chamber.

36. The device of claim 35, wherein the one or more channels in the walls of the access port enter the chamber at different heights.

37. A bioreactor device comprising:
a hard layer defining one or more cell culture chambers, the one or more cell culture chambers comprising an inlet opening and an outlet opening at a base of the cell culture chamber; and
a soft layer for mounting to the hard layer, the soft layer defining one or more openings to receive with the one or more cell culture chambers and inlet and outlet flow channels that align with the inlet and outlet openings at the base of the cell culture chamber.

38. The device of claim 37, wherein each cell culture chamber is connected to the other cell culture chambers by an inlet and outlet flow channel.

39. A method of culturing one or more cell types in a three-dimensional environment comprising culturing the one or more cell types in a bioreactor device of any one of claim 1, 33, or 37.

40. The method of claim 39, wherein one of the one or more cell types is a cancer cell.

41. The method of claim 40, wherein the one or more cell types further comprises epithelial cells, fibroblasts, adipocytes, endothelial cells, tumor associate macrophages, T-cells, B-cells or a combination thereof.

42. The method of claim 40, wherein the cancer cell is a breast cancer cell, a lung cancer cell, ovarian cancer cell, a pancreatic cancer cell, or a glioblastoma.

43. A method for assessing responsiveness to therapeutic agents comprising:
culturing in a three-dimensional environment a plurality of cell samples in a plurality of wells or sub-wells of the bioreactor devices of any one of claim 1, 33, or 37;
exposing each cell sample in a well to a different therapeutic agent or a different concentration of the same therapeutic agent by perfusing media through the plurality of chambers comprising the appropriate concentration of therapeutic agent; and
measuring the responsiveness to each cell sample to the therapeutic agent.

44. The method of claim 43, wherein the cell sample is a cancer cell sample.

45. The method of claim 44, wherein the cancer cell sample is a obtained from a biopsy sample of a subject in need of therapeutic treatment.

46. The method of claim 45, wherein the cancer cell sample is a breast cancer sample, ovarian cancer sample, a pancreatic cancer sample, a lung cancer sample, or a glioblastoma.

47. The method of claim 43, wherein the plurality of cell samples is cultured in a microtumor model comprising epithelial cells, fibroblasts, adipocytes, endothelial cells, tumor associate macrophages, T-cells, or B-cells or a combination thereof.

48. The method of claim 43, wherein responsiveness is measured by observable phenotypic changes in cell structure on the device, the release of one or more biomarkers in the perfused media, or through lysis and detection of one or more biomarkers.
